(19) United States
(12) Patent Application Publication
(10) Pub. No.: US 2023/0298688 A1
(43) Pub. Date: Sep. 21, 2023

(54) MICROBIOME ANALYTICS SUCH AS FOR ANIMAL NUTRITION MANAGEMENT

(71) Applicant: CAN TECHNOLOGIES, INC., Wayzata, MN (US)

(72) Inventors: Vernon L. MCINTOSH, Minneapolis, MN (US); Jean E. DE OLIVEIRA, Vilvoorde (BE)

(73) Assignee: CAN TECHNOLOGIES, INC., Wayzata, MN (US)

(21) Appl. No.: 18/000,161

(22) PCT Filed: May 27, 2021
(86) PCT No.: PCT/US2021/034499

§ 371 Date: Nov. 29, 2022

Related U.S. Application Data
(60) Provisional application No. 63/032,376, filed on May 29, 2020.

(51) Int. Cl. G16B 20/00; G16H 50/20; G16B 40/20

(52) U.S. Cl. CPC G16B 20/00; G16B 40/20; G16H 50/20

(57) ABSTRACT

A method of training a microbiota model engine to identify biomarkers for predicting food safety or animal growth includes obtaining data that is indicative of an assay of candidate biomarkers obtained the gastrointestinal tracts of a set of animals, where the assay is performed at specified intervals in the lifecycle of the animals and the animals manifest specified characteristics at the specified intervals. The method further includes training the microbiota model engine using the data to generate a prediction based on at least one of a food safety or an animal growth criterion and obtaining, from the trained microbiota model engine, a set of features used by the microbiota model engine to generate the prediction. The method additionally includes identifying a subset of biomarkers from amongst the candidate biomarkers from the set of features and providing the subset of biomarkers for generating food safety or animal growth predictions.

### CROSS REFERENCE TO RELATED APPLICATIONS

This application claims the benefit of U.S. Provisional Pat. Application No. 63/032,376, filed May 29, 2020, and entitled “MICROBIOME ANALYTICS SUCH AS FOR ANIMAL NUTRITION MANAGEMENT”, which is incorporated by reference herein in its entirety.

### TECHNICAL FIELD

Embodiments of the present disclosure relate generally to agriculture, and more particularly, but not by way of limitation, to predicting or controlling pathogen risk and animal growth using machine learning and gastrointestinal microbiome composition.

### BACKGROUND

Animals, such as livestock, maintain a delicate balance between a diverse collection of bacteria, viruses, protozoa, fungi, and other microorganisms and associated genetic material in their gastrointestinal tracts (GIT). These microorganisms and associated genetic material (hereinafter, “microbiota”) collectively constitute the gut microbiome of some animals. Maintaining a stable gut microbiome, or a balanced community of microbiota, in the gastrointestinal tract of an animal can be important for the animal’s ability to digest feed products and for the overall performance of the animal as livestock. Maintaining a stable gut microbiome in livestock animals is also beneficial to the health of consumers, as some microbiota that are present in the microbiome may also be present in consumable products produced from the animals. A subset of microbiota that are present in consumable products include certain bacteria or viruses which, while being innocuous to the host animal, may pose downstream risks from a food safety perspective. Sustaining such a balance in animals that are raised as livestock can be difficult due to, for example, the constant influence of interacting factors, such as the environment where the animals are raised, the feed or nutrients that the animals consume, and competition between microbiota species or populations for substrate and other resources.

### BRIEF DESCRIPTION OF THE DRAWINGS

Various ones of the appended drawings merely illustrate example embodiments of the present disclosure and cannot be considered as limiting its scope.

FIG. 1 is a diagram illustrating an example of a system to control animal performance or pathogen risk using machine learning and GIT microbiota data, according to various embodiments.

FIG. 2 is a diagram illustrating an example of a system to generate a database for training or operating a system to control animal performance or pathogen risk using machine learning and GIT microbiota data, according to various embodiments.

FIG. 3 is a diagram illustrating an example of a system to train a microbiota engine for use in a system to control animal performance or pathogen risk using machine learning and GIT microbiota data, according to various embodiments.

FIG. 4 is a diagram illustrating an example of a system for using a microbiota engine to process GIT microbiota data using machine learning, according to various embodiments.

FIG. 5 depicts an example of a process for training a microbiota engine to identify biomarkers for predicting food safety or animal growth, according to various embodiments.

FIG. 6 depicts an example of a process for using a microbiota engine to predict the performance or pathogen risk of a set of one or more animals based on GIT microbiota, according to various embodiments.

FIG. 7 depicts an example of a process for using a microbiota engine to control the performance or pathogen risk of a set of one or more animals based on GIT microbiota, according to various embodiments.

FIG. 8 depicts an example of a process for operating a system to control animal performance or pathogen risk using machine learning and GIT microbiota data, according to various embodiments.

FIG. 9 is a diagram illustrating an example of a user interface reporting animal performance or pathogen risk based on GIT microbiota, according to various embodiments.

FIG. 10 is a diagram illustrating an example of a user interface reporting a graphical data that is indicative of animal performance or pathogen risk based on GIT microbiota, according to various embodiments.

FIG. 11 illustrates a diagrammatic representation of a machine in the form of a computer system within which a set of instructions may be executed for causing the machine to perform any one or more of the techniques discussed herein, according to various embodiments.

The headings provided herein are merely for convenience and do not necessarily affect the scope or meaning of the terms used.

### SUMMARY OF THE DISCLOSURE

An example of the present disclosure includes techniques for training a microbiota model engine to identify biomarkers for predicting food safety or animal growth. The techniques can include obtaining data that is indicative of an assay of candidate biomarkers obtained the gastrointestinal tracts of a set of animals, where the assay is performed at specified intervals in the lifecycle of the animals and the animals manifest specified characteristics at the specified intervals. The techniques can further include training the microbiota model engine using the data to generate a prediction based on at least one of a food safety or an animal growth criterion and obtaining, from the trained microbiota model engine, a set of features used by the microbiota model engine to generate the prediction. The techniques can additionally include identifying a subset of biomarkers from amongst the candidate biomarkers from the set of features and providing the subset of biomarkers for generating food safety or animal growth predictions.

Another example of the preset disclosure includes techniques for determining a model that can be used for classification or predictions for an animal based on microbiota data. The techniques can include obtaining first data that is indicative of genetic material of first microbiota obtained from a gastrointestinal tract of an animal at specified intervals in the lifecycle of the animal. The techniques can further include determining, based on the first data and using a first microbiota model engine, a model for the animal, the first microbiota model engine trained using supervised learning and data obtained from gastrointestinal tracts of two or more animals. The techniques can additionally include providing the classification in a computer readable data structure for display on a graphical user interface.

Another example of the present disclosure includes techniques for reducing antibiotic usage to control the presence of a pathogen in a population of animals. The techniques can include determining, using a microbiota model engine that is stored in the memory of a computing system, a set of biomarkers from gastrointestinal tracts of the animals that are indicative of the presence of the pathogen. The techniques can also include obtaining first data that is indicative of assay of candidate biomarkers of first microbiota obtained from a gastrointestinal tract of an animal at specified intervals in the lifecycle of the animal. The techniques can further include identifying, using the set of biomarkers and the first data, an additive to a feed product of the animals for adjusting a presence of the pathogen. The techniques can additionally include adjusting a quantity of the additive in the feed product to reduce the presence of the pathogen.

Another example of the present disclosure includes graphical user interface (GUI) to report a sample analysis, the GUI comprising. The GUI can include a first area to report a summary of the analysis and a second area to report a graphical categorical metric associated with the summary of the analysis.

Another example of the present disclosure includes a GUI to report a sample analysis of a population of animals. The GUI can include a first area to report a current distribution of microbes in a population, a second to report a predicted distribution of microbes in the population, and a third to report a financial impact associated with the current or predicted microbial distribution.

### DETAILED DESCRIPTION

The description that follows includes techniques (e.g., systems, methods, instruction sequences, and computing machine program products) that embody illustrative embodiments of the disclosure. In the following description, for the purposes of explanation, numerous specific aspects are set forth in order to provide an understanding of various embodiments of the inventive subject matter.

Examples of the present disclosure are based on the inventors’ recognition that farm animal performance and pathogen risks and can be improved through the identification of relationships between populations of GIT microbiota and by techniques that link animal nutrition to GIT microbiota based on these identified relationships. Existing techniques for acquiring and processing GIT microbiota data may only afford isolated sampling at irregular intervals, such as can make it difficult to gain insight into the microbiome of a population of animals on an ongoing basis. In an example, research into GIT microbiota may require large volumes of data which can be prohibitively expensive to generate using generally available techniques. Although such data can, in some situations, be aggregated from disparate sources, such as public repositories or consortia, the usefulness of this aggregated data can be limited, such as due to variation in the methodology and competency with which each source generates their data. Additional sources of variation in generated or aggregate GIT microbiota data can arise from variations in the several factors that influence the microbiome. Such factors can include the host animal genetics, the environment in which the animal is reared, the rearing history, and the diet of the host animal and the initial GIT colonization or composition of the host animal. The variability caused by these factors and the disparate sources of aggregate GIT microbiota data can introduce noise or confounding factors, which can limit an ability to obtain useful insight though existing manual or computer-assisted analysis techniques.

Examples of the present disclosure overcome the limitations of existing techniques for acquiring and analyzing GIT microbiota data, such as in part by providing techniques for identifying a reduced set of GIT microbiota biomarkers (hereinafter, “biomarkers”) that are useful for generating predictions for specified topics of animal performance pathogen risk. Such reduced data sets are established in part using a machine learning technique to identify select biomarkers predictive of specified attributes such as animal performance or pathogen risk. In an example, the techniques of the present disclosure include obtaining a database that associates GIT microbiota data of a set of animals with microbiome, animal performance, and pathogen risk data of the set of animals at specified stages or times during the lifecycle of the animals. The GIT microbiota data includes deoxyribonucleic acid (DNA) or sequences or ribonucleic acid (RNA) sequences (hereinafter, “genetic data” or “genetic information”) that are associated with, or indicative of, GIT microbiota in the digestive tracts of the set of animals. The microbiome, animal performance, and pathogen risk data can include data that is indicative of any factor or piece of information that is associated with the animal microbiome composition, performance, or pathogen risk. In an example, this database includes data that is indicative of the genetics, rearing environment, rearing history, diet, and the initial GIT colonization or composition of the animals. Such data can also include data that is indicative of the weight, size, or chemical composition of the animal. In an example, the database includes data for substantially the entire microbiota population of the animals. The database can be used to identify or select, for each topic of interest using machine learning techniques, a set of biomarkers for making a specified prediction for the topic. In an example, the database is used to train a machine learning model to generate the prediction, while the set of biomarkers are extracted from the trained model, such as by identifying principal features used by the model to make a prediction.

Examples of the present disclosure overcome the limitations of existing techniques for acquiring and analyzing GIT microbiota data by providing techniques that reduce the amount of data used to classify, or make predictions about, animals based of GIT microbiota. In an example, while existing microbiota analysis techniques may capture and analyze the totality of data available for all a microbiome to generate a prediction or to obtain certain insights, the techniques of the present disclosure include selecting a target topic of interest, identifying biomarkers that are suitable for generating predictions regarding the topic of interest, and only capturing and analyzing data associated with the identified biomarkers to make a prediction regarding the topic. The identified biomarkers can correspond to specific GIT microbiota and generally constitute small subset of biomarkers represented in a microbiota assay or a microbiota database.

Examples of the present disclosure provide techniques for using GIT microbiota and machine learning to reduce antibiotic usage to control the presence of a pathogen in a population of animals.

Examples of the present disclosure provide techniques for using GIT microbiota and machine learning to reduce antibiotic usage to identify or predict feed additives that that can improve animal performance.

Examples of the present disclosure provide a graphical user interface for reporting sample analysis of a population of animals.

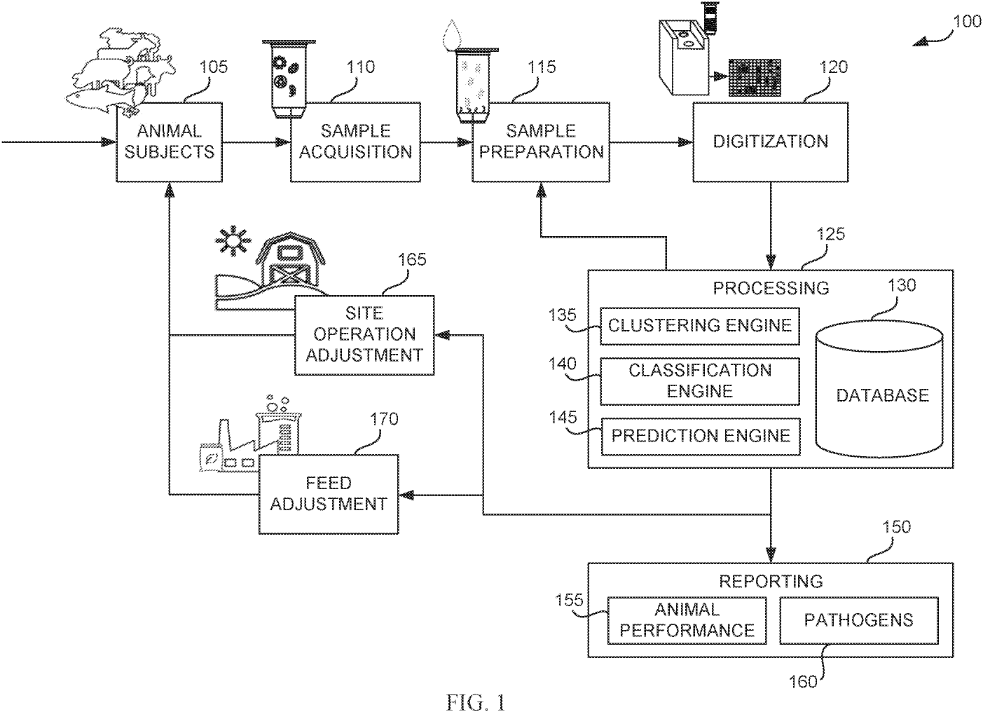

Turning now to the figures, FIG. 1 is a diagram illustrating an example of a system 100 to control animal performance or pathogen risk using machine learning and GIT microbiota data, according to various embodiments. The system 100 can implement any of the techniques described herein. The system 100 can include animal subjects component 105, sample acquisition component, 110, sample preparation component 115, digitization component 120, processing component 125, reporting component 150, site operation adjustment component 165, and feed adjustment component 170. In an example, the system 100 includes one or more of these components. In other examples, the system 100 includes outputs, data, or other information or materials that are derived from one or more of these components.

Animal subjects component 105 can include one or more groups of livestock, farmed animals, or another other wild or domesticated animal (hereinafter, “animals” or “animal subjects”). In some examples, animal subjects component 105 includes flocks of birds, schools of fish, drifts or droves of pigs, or herds of cows, goats, or sheep. The animal subjects component 105 can include animals that are bred, reared, or otherwise cultivated at a single location or site, such as in an aquarium, at farm, in field, or in laboratory. The animal subjects component 105 can also include a collection of animals from disparate sites, locations, or geographic areas (hereinafter, “farm sites”). In certain examples, the animal subjects component 105 includes techniques, and other information that are associated with a farm site or particular methods employed by the farm site to breed, rear, or cultivate animals.

Animal subjects component 105 can include techniques (e.g., methods, systems, devices, or processes) used by a farm site, or by an animals products processing facility, to characterize the physical attributes of animals. In an example, the animal subjects component 105 includes techniques for determining the performance of an animal. Such performance can include weight, chemical body composition, nutrient content, growth rate, production efficiency or any other suitable metric for evaluating the economic or utilitarian value of an animal. In another example, the animal subjects component 105 includes techniques for determining whether an animal manifests signs of an illness and for identifying a macro cause of such illness, such as a microbial imbalance including bacteria or virus.

Animal subjects component 105 can include any information, or means for acquiring information, that is associated with the aforementioned aspects of the animal subjects component.

Sample acquisition component 110 can include any suitable system or technique for obtaining a set of biological samples from animals. The sample acquisition component 110 can be configured to obtain the biological samples from the GIT of animals from one or more farm sites. In an example, the biological samples are obtained using a permeable material or substrate, such as swab, sponge, or other material that is configured to wipe and secure biological material (e.g., digesta such as chyme or excreta such as droppings of the animals) from a surface or orifice of the GIT of the farm animals. In another example, the biological samples are taken using a non-permeable material, such as a glass or polymer tube, vial, or other container that is configured to receive the biological samples directly from the animals or from an intermediary sample acquisition component. In another example, the biological sample is content obtained from a segment of the GIT of the animals, such from the ileum or cecum of processed animals. In yet another example, the biological samples are obtained from exposed orifices of animals or from droppings produced by the animals.

In an example, the sample acquisition component 110 includes a standardized sample acquisition assembly (e.g., a sample kit) that includes a glass or polymer tube, a chemical solution or reagent, and one or more swabs. The tube can be pre-filled with the chemical solution. In example, the chemical solution includes a solution that is configured to lyse microbiota cells and preserve DNA/RNA. Such solution can include DNA/RNA Shield™ produced by Zymo™ or another suitable solution. In an example, the sample acquisition assembly includes prescribed sample collection acquisition and handling protocols. Such protocols can include directions regarding the number of samples to obtain per farm site, a process for collecting and storing each sample, a process for shipping the samples, and a process for pooling collected samples for analysis. The sample acquisition assembly, including the described protocols, can standardize the sample acquisition process and thereby reduce variations in microbiota genetic data obtained from the samples.

The sample acquisition component 110 can be configured to obtain GIT biological samples (hereinafter, “samples”) from one or more populations of animals from one or more disparate farm sites. In an example, the samples are obtained from different species of animals, animals within the same species that are cultivated for different purposes, animals from different geographic areas, animals fed different diets, different ages or animals housed or reared in different environments. The sample acquisition component 110 can also be configured to obtain samples from a specific population of animals, such as a target flock of birds at a selected farm site.

Sample preparation component 115 can include any suitable system or technique for preparing a biological sample obtained from the GIT of animals for digitization, such as for generating a microbiome functional or composition diversity data set (hereinafter, “sample data set”) based on the biological samples. Such sample data set can include any data that is indicative of genetic sequences or markers that are obtained from, and suitable for identifying, microbiota included in the biological sample. Such data can be represented in any suitable format, such as operational taxonomic units (OTUs), 16S sequences, 18S sequences, internal transcribed spacer (ITS) sequences, or as any other suitable genetic markers. Such data can include information that is indicative of the relative abundance, diversity, or distribution of microbiota of given taxonomic ranks in the microbiome of the farm animal from which the biological sample is obtained. In certain examples, the sample data set can include data that is indicative of metabolites detected in the biological sample, such as to identify functional aspects of a microbiome, such as selected metabolic pathways or catalytic activity. Accordingly, the sample preparation component 115 can include any suitable technique or device for preparing a biological sample to extract this information. Processing a biological sample using existing techniques, however, can be financially expensive and may produce such large volumes of genetic or metabolic information that analyzing the data sets obtained from these samples to obtain useable insights may be an intractable problem.

In an example aspect of the present disclosure, the sample preparation component 115 includes a specially fabricated DNA or RNA microarray chip (hereinafter, “array chip”) that is configured to selectively process a biological sample based on a set of predetermined biomarkers. Such biomarkers include a selected set of microbiota genetic or metabolic material whose presence in the biological sample is indicative of a topic or phenomenon of interests, such as the performance, intestinal health, or pathogen risk of a farm animal. Such biomarkers can be used to limit the scope (number or formatting) of bacteria lists obtained by other identification and quantification techniques in order to enter the method hereby described. In an example, a selected set of biomarkers is associated with a particular microorganism or a taxonomic rank of microorganisms. In an example, a selected set of biomarkers are associated with specific bacteria, such as Salmonella enterica or Lactobacilli crispatus. In an example, a selected set of biomarkers are associated with specific genus of bacteria, such as Alistipes, Bacteroides, Bifidobacteria, Campylobacter, Erysipelotrichaceae, Faecalibacterium, Lactobacilli, Ruminococcus, Salmonella, Streptococcus, Clostridium, proteolytic bacteria, or any subtaxa or organism thereof. Configuring the array chip can include selecting a suitable set of biomarkers based on a topic of interest (e.g., an investigative purpose for which the biological sample was obtained), identifying one or more probes (e.g., oligonucleotides such as a polymerase chain reaction primer/probe) for amplifying and detecting genetic material that is indicative of the biomarkers, and fabricating an array chip using the identified probes. In an example, the array chip is integrated to into a glass tube, such as an array tube.

In an example aspect of the present disclosure, the sample preparation component 115 includes techniques for obtaining microbiota genetic material from the sample acquisition component 110 and exposing the genetic material to the array chip in an array tube comprising any suitable assay reagents.

The digitization component 120 can include any suitable thermal cycler or DNA amplifier (e.g., a PCR machine) that is configured to amplify the exposed DNA material using the probes of the array chip and any suitable genetic material amplification technique. A result of such processing is amplified genetic material corresponding to the genetic material of microbiota associated with the selected set of biomarkers. A florescent signal generated by the amplified genetic material be read by the digitization component 120 and used to generate the sample data set. The use of probes derived from selected biomarkers enables the amplification of corresponding genetic material that are present in such small quantities in the biological sample that such material may not be detectable of using other sample preparation techniques. Additionally, the use of probes derived from the selected biomarkers improve the likelihood that the amplified genetic material primarily or only contains the genetic material of microbiota associated with selected set of biomarkers, thereby reducing the number of salient signals, or the amount of information, that is be obtained from the biological sample to those of interest.

Processing component 125 can include any computing resource (e.g., computing system, computing environment, or partition of a computing environment that is allocated to a user of a computing resource) that is configured to process a sample data set as described herein. In an example, the processing component 125 includes database 130, clustering engine 135, characterization engine 140, and prediction engine 145.

The database 130 includes a microbiota database that includes data that is indicative of biomarkers, DNA or RNA sequences, or other genetic information (hereinafter, “biomarker information”) of microbiota obtained from biological samples acquired from the GIT of animals at one or more stages or times during the lifecycle of the animals. In an example, the database 130 includes data that is indicative of biomarkers obtained from biological samples of flocks of birds, such as gallus gallus domesticus (e.g., broiler chicken), where the samples are obtained are acquired periodically (e.g., every 2 days) throughout the life cycle of the birds. The database 130 can include information that is indicative of the presence of absence of the biomarkers in the biological samples and information that is indicative of the quantity of biomarkers present in the samples (e.g., the strength of signals generated by probes designed to detect the biomarkers). The database 130 can also include, and associate the biomarkers with, supplementary information, such as the date on which a sample was acquired, the type of animal from which the sample was acquired, the geographical location of the animal, the particular farm or site where the animal is reared, the feed provided to the animal, the types nutrient additives provided to the animal or added to the feed, or the physical characteristics of the animal (e.g., age, physical size, or weight). The database 130 can also include supplementary data that is indicative of the health of the animal when the sample is acquired.

In an example, the database component 130 is populated with biomarker information and associated supplementary information that is obtained according to the sample acquisition, preparation, and digitization techniques described herein.

In an example, the database 130 includes biomarker information and associated supplementary information obtained from at least 10,000 biological samples obtained from flocks of chickens.

The processing component 125 can also include one or more machine learning components, such as clustering engine 135, classification engine 140, and prediction engine 145. Each of the machine learning components can include one or more machine learning models that are trained according to one or more supervised or unsupervised machine learning algorithms and using the biomarker information and supplementary information stored in the database 130. Examples of suitable supervised machine learning algorithms can include Bayesian networks, decision trees, K-nearest neighbors, linear classifiers, linear regression, logistic regression, naive Bayesian algorithms, quadratic classifiers, random forests, support vector machines, and other suitable algorithms. Examples of suitable unsupervised machine learning algorithms include expectation-maximization algorithms, vector quantization, and information bottleneck methods.

One or more machine learning components can be used to identify features (e.g., any individual measurable property or characteristic of the database 130 that used in the execution or evaluation of a machine learning model), or to select data, from information stored in the database 130 for training another machine learning component. In an example, the clustering engine 135 can include one or more unsupervised machine learning models that are trained using data from the database 130, such as data that has been preprocessed using dimensionality reduction techniques such as principal component analysis, to identify features from the database that exhibit a latent relationship. Such features can then be used to select training data sets (e.g., data sets including pairs on input and output data) for training supervised machine learning models.

In an example, clustering engine 135 can be trained and used to select a reduced set of biomarkers from the database 130 for identifying microbiota whose presence in the GIT of an animal is associated with, or predictive, of a topic of interests. Such biomarkers can then be used to select data from the database 130 to train classification engine 140 or predictive engine 145 and to fabricate array chips for selectively obtaining biomarker data from biological samples for providing inputs to these engines. In an example, the clustering engine 135 can be used to identify a set of biomarkers that are suitable for identifying microbiota whose presence in the GIT of a flock of chickens animal is associated with, or predictive, of the performance or pathogen risk of the chickens.

The classification engine 140 can be configured with one or more trained machine learning models that are configured to classify one or more data sets of the database 130 according to one or more categories based on a topic of interest. In an example, the classification engine 140 can be used to classify information in database 130 into one or more categories based on whether the biological samples from which the information was obtained included certain types or groups of microbiota or any other feature of the database. In an example, the classification engine 140 can be used classify information in database 130 based on whether biological samples from which the data was obtained had a strong Salmonella, Campylobacter, or other bacteria presence. Such classifications can be based on identifying a threshold signal strength for one or more biomarkers for determining whether to attribute the data in the database to one category or another. Machine learning can be used to identify the set of biomarkers that are most responsive to the classification inquiry or to determine the threshold signal strength. In an example, the clustering engine 135 can be used to identify the suitable biomarkers for the classification, while a predictive engine, such as a regression model, can be used determine suitable threshold signal strengths.

The prediction engine 140 can be configured with one or more trained machine learning models that are configured to make predictions regarding a topic of interest. In an example, the prediction engine 140 is trained using supervised learning techniques. In another example, the prediction engine 140 is configured with one or models that are trained to correlate the presence (e.g., quantity) or absence of particular microbiota with a physical characteristic of a selected animal, such as weight or size (hereinafter, “animal performance”) at specified stage or time in the life cycle of the animal. Such trained models can be used to predict the weight of an animal based on a biological sample obtained from the animal. In an example, such training includes identifying one or more sample times in the life cycles of the taxonomic rank or group to which the selected animal belongs that are most predictive of the physical characteristic or animal performance topic. In an example, the identified sample times can include any sample time associated with the lifecycle of animals in the group, including, for example, any time from birth or hatching of the animals up to and including a stage when the animals are aged to correspond to the age of the selected animal at the specified time. In some examples, the identified sample times can include sample times prior to the birth or hatching of the animals, such as to include samples obtained from a progenitor of the selected animal prior to the selected animal’s birth.

Data from the identified sample times can then be used to train the predictive models. In an example, a predictive model that is suitable for predicting the weight of a selected animal at 35 days into the life cycle of the animal based on a biological sample obtained from the animal at 21 days in the lifecycle of the animal can be trained by identifying data, include supplementary data that is indicative of animal weight, from all samples in the database 130 for animals that belong to the same group as the selected animal that were obtained from samples taken 21 days into animals’ life cycle and training the models using the obtained. Such training can include using biomarker information as input and supplementary data that is indicative animal weight as output in a supervised learning training data set. The identified sample times can be determined using machine learning techniques, such as by generating one or more predictive models using data from the database 130 that is obtained at different sample times and comparing the predictive efficacy of each model. In some examples, the dimensions of the sample data set can be reduced by using machine learning to identify biomarkers that are most predictive of the target animal performance metric.

In an example, the prediction engine 140 is configured with one or models that are trained to correlate the presence or absence of particular GIT microbiota in animals with a type feed or a nutrient additive provided to the animals, such as by using a training technique that is substantially similar to the previously described technique. Such trained models can be used predict or identify a nutrient that can control the presence of the particular microbiota in the GIT of selected animals over time. This information can be used to identify novel feed ingredients or to adjust feed formulations so as to limit pathogen risk.

In an example, the prediction engine 140 is configured with one or models that are trained to correlate the presence or absence of a first type of microbiota with the presence of absence of a second type of microbiota, such as by using a training technique that is substantially similar to the previously described technique. Similar to predicting the future weight of an animal, the trained models can be configured to predict the future presence of the second type of microbiota based on the presence of the first type of microbiota. Such models can be used to formulate intervention strategies, such as changes in nutrient additives or farm management, to limit the development of the second type of microbiota so as to, for example, avoid the development of pathogens that pose a pathogen risk. Such techniques can be used to reduce antibiotic usage in animals.

The machine learning component of the processing component 125 can include any other components or techniques that are suitable for classifying or predicting performance or health characteristics of groups of one or more animals and farm sites based on GIT microbiome where such predictions are based on trained machine learning models that are derived from a database of GIT microbiome data or analysis generated from GIT biological samples obtained at one or more stages or time points during the life cycle of such animals or of animals reared at such farm sites.

The machine learning component of the processing component 125 can include any other components or techniques that are suitable for identifying feed, feed additives, nutrients or farm management practices that can affect or control the presence or absence of one or more types of GIT microbiota in farm animals, where such identifying is based on trained machine learning models derived from a database of GIT microbiome data or analysis generated from GIT biological samples obtained at one or more states or time points during the life cycle of such animals.

The machine learning component of the processing component 125 can include any other components or techniques that are suitable for identifying a reduced or minimum set of biomarkers that are suitable for characterizing the GIT microbiome of animals according to a topic of interests or for obtaining training or operating machine learning models to implement any of the techniques described herein, where such identifying is based on trained machine learning models derived from a database of GIT microbiome data or analysis generated from GIT biological samples obtained at one or more stages or times during the life cycle of such animals. In an example, the reduced set of biomarkers are obtained or extracted from features of one or more machine learning models. In another example, the reduced set of biomarkers are used to determine primers or probes for fabricating an array chip, as described herein.

The reporting component 150 can include any computing resource that is configured to provide data, classifications, predictions, recommendations, or analysis that are derived from the operation of the processing component 125 or any component thereof. In an example, the reporting component 150 includes an internet or web server, a website hosted by an internet or web server, or a software application. The reporting component can include animal performance component 155 and pathogens component 160. The animal performance component 155 can provide data, classifications, predictions, or recommendations associated with the performance of a group of one or more animals based on the GIT biological samples obtained from the animals. The pathogens component 160 can provide data, classifications, predictions, or recommendations associated with the presence, absence, or predicted emergence of pathogens, such as pathogens that are associated with pathogen risk, in a group of one or more animals based on the GIT biological samples obtained from the animals.

Site operation adjustment component 165 can include any suitable techniques for adjusting the operation of a farm site to improve the health or performance of animals based on classifications or predictions obtained from the processing component 125 or the reporting component 150. In an example, such techniques include adjusting feeding strategies (e.g., feeding schedule, feed composition, etc.) or the environment to control the growth or development of animals or an offspring of the animals, such as by adjusting the GIT microbiome component of the farm animal.

Feed adjustment 170 can include any suitable techniques for adjusting the feed or nutrients of animals to improve the pathogen risk or performance of animals based on classifications or predictions obtained from the processing component 125 or the reporting component 150. In an example, such techniques include identifying existing or novel ingredients or nutrients to add to the feed of animals to adjust the presence of absence of microbiota in the GIT of the animals. In another example, such techniques include identifying existing ingredients or nutrients to change (e.g., adjust in concentration or volume) in the feed of animals to adjust the presence of absence of microbiota in the GIT of the animals. Adjusting the feed or nutrients include changing the amount of one or more ingredients or nutrient additive or changing recommended portion sizes or feeding schedules.

In an example operation, a database, such as the database 130, of GIT microbiome information that is indicative of animal performance and GIT health is generated based on GIT biological samples obtained from farm animals, such as a flock of chickens, at different stages in their life cycles. A set of biomarkers are identified using the database 130 and one or more machine learning techniques. In an example, biomarkers are selected based a topic of interest, such as animal performance or pathogen risk. In another example, the biomarkers are suitable for identifying a selective set of microbiota in GIT biological samples of the farm animals for obtaining classification or predictive information about the topic (e.g., information or predicting animal performance or pathogen risk based on the GIT biological sample). The selected biomarkers are used to fabricate an array chip that is suitable for obtaining the classification or predictive information. The array chip is used to process GIT biological samples from the farm animals to obtain GIT microbiome composition information. The GIT microbiome composition information is used with trained machine learning models to provide classifications or predictions based on the topic of interest. The classifications or predictions are then used to adjust farm site operations or feed development or provision strategies to control the performance or pathogen risk of animals.

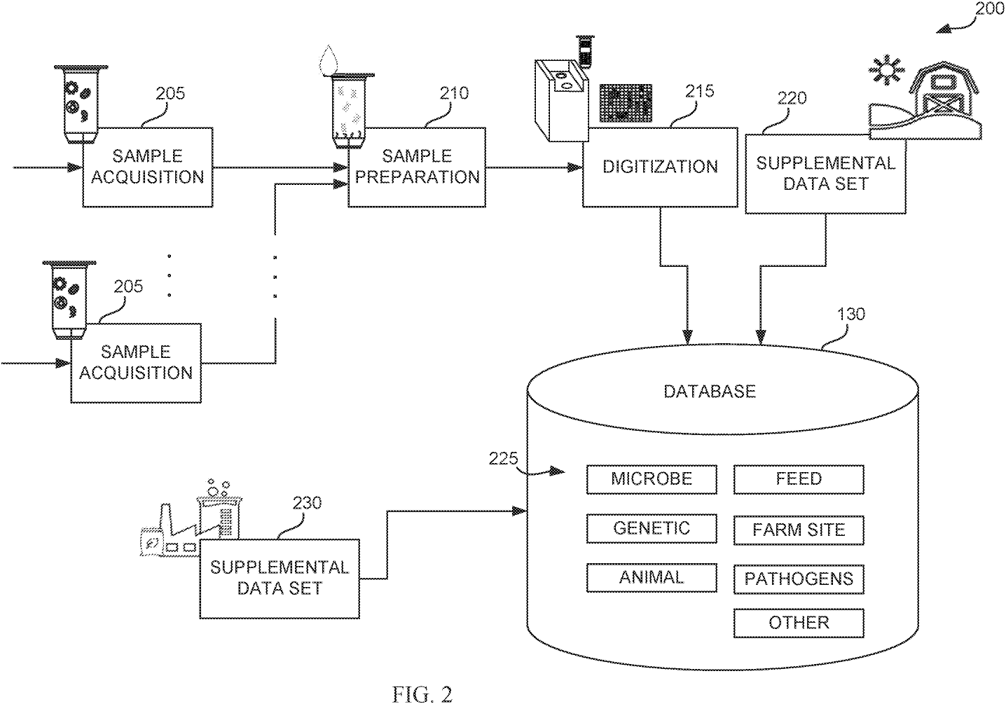

FIG. 2 is a diagram illustrating an example of a system 200 to generate the database 130 (FIG. 1) for training or operating a system to control animal performance or pathogen risk using machine learning and GIT microbiota data, according to various embodiments. As shown in FIG. 2, the system 200 includes sample acquisition component 205, sample preparation component 210, digitization component 215, and supplementary data sets 220 and 230. The sample acquisition component 205, sample preparation component 210, and digitization component 215 correspond to and perform similar functions as, the sample acquisition components 110, sample preparation component 115, and digitization component 120 as shown in FIG. 1. Such components are configured to acquire GIT biological samples from animals at one or more farm sites, process and digitize the samples and provided the digitized information for storage in the database 130. The supplemental data set 220 and 230 can include any techniques or components for obtaining and associating farm site or feed processing facility information with the acquired GIT biological samples or the animals that generated the biological samples. The supplemental data set 220 and 230 can include any farm site or feed processing or composition information that is associated with acquired GIT biological samples or the animals that generated the biological samples.

The database 130 can include data 225 obtained from the GIT biological samples and supplemental data 220 and 230. In an example, the database 130 includes microbe, genetic, animal, phenotype, age, feed, geography, pathogen, farm site, or any other animal or GIT microbiome related data described herein. The microbe data can include any data that is suitable for identifying, classifying, or quantifying the presence or absence of, one or more taxa of microbes. The genetic data (e.g., gene sequences, gene loci, etc.) can include any data that is indicative of or associated with genetic information obtained from a GIT biological sample of an animal. The animal data can include any data that is associated with a physical condition of an animal, such as the birth date, age, size, weight, taxa, health, disposition, or lineage of the animal. The feed data can include any data that is associated with animal feed, nutrients, or other ingredients or substances provided to animal during its life cycle. The farm site data includes any data that is associated with a farm site where an animal is reared, such as geographical location, political designation (e.g., country), or climate or other environmental conditions. The farm site data can also include any suitable data that is indicative for the operations or practice of a farm site. The pathogen data can include any data that is indicative of pathogens that affect the health of animals or consumers.

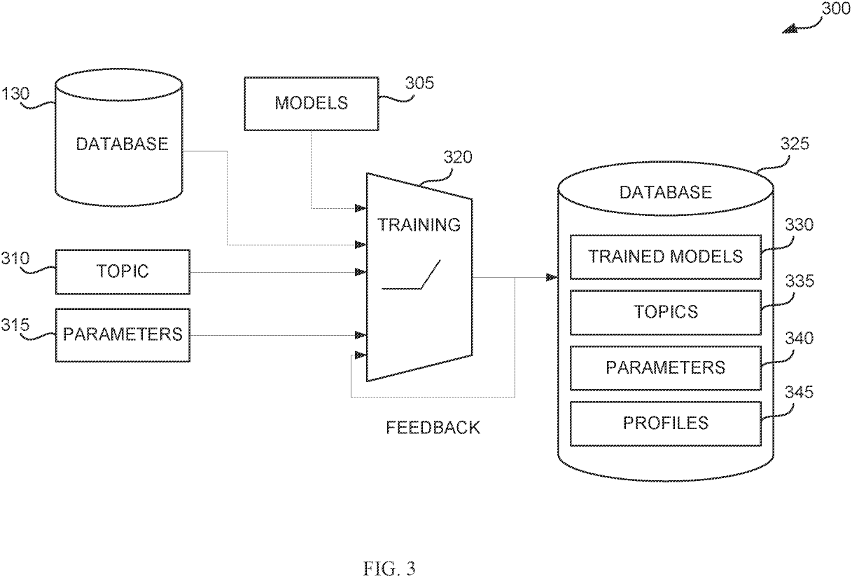

FIG. 3 is a diagram illustrating an example of a system 300 to for generating an engine for use in a system to control or predict animal performance or pathogen risk using machine learning and GIT microbiota data, according to various embodiments. The system 300 can include the database 130, machine learning models 305, topics 310, parameters 315, training component 320 and database 325.

Training component 320 is configured to train the machine learning models 305 using GIT microbiota data from the database 130 according to one or more supervised or unsupervised machine learning techniques. In an example, the microbiota is preprocessed, such as by labeling one or more data sets to form data sets comprising input-output pairs that are usable in supervised machine learning. In an example, such labeling includes labeling genetic information included in the GIT microbiota data to associate the genetic information with specific GIT microbiota or microbiota taxa. Such labeling can also include labeling genetic information included in the GIT microbiota data to associate GIT microbiota with one or more pathogen risks or indicators of pathogen risk. The models 305 can include one or more machine learning clustering, classification, or prediction models, such as the models used to generate the clustering engine 135, the classification engine 140, or the prediction engine 145.

In an example, the training component 320 can train the models 305 using an iterative learning process whereby input-output pairs from labeled portions of the microbiota data is used to learn one or more model parameters for generating one or more trained models 330 that are configured to perform the clustering, classification, or prediction operations described herein. Such training can include identifying or generating suitable loss functions for measuring or characterizing the distance, or difference, between an output generated by a model in response to a provided input and a labeled output that is associated with the provided input. Such training can also include providing a feedback or a feedforward path to for adjusting the training or the parameters of the model based on the determined distance.

In an example, the training component 320 can use one or more topics 310 and associated parameters 315 to improve or adjust the training. In an example, the topics 310 can include information for model selection or for preprocessing the GIT microbiota data, such as to preselect models that are likely to be suitable for responding to inquiries associated with a selected topic. In another example, the parameters 315 can be used by the training component 320 to identify one or more thresholds, rules, or other criteria for preprocessing the GIT microbiota data, selecting models 305, developing loss functions for the models, or for evaluating the performance of the models. In an example at the topics 310 or the parameters 315 can be learned or inferred by the models 305 or the training component 320 so to generate learned topics 335 and learned parameters 340. The learned topics 335 or the learned parameters 340 can include the topics 310 or the parameters 315.

In an example, the trained models can be associated with, or categorized into one or more groups based on the learned topics or learned parameters 340. The learned topics 335, or any other topics described herein, can include any animal performance topic (e.g., animal weight, size, nutrition level, etc.) or pathogen risk topic. The learned parameters 340, or any other topics described herein, can include any criteria or threshold value for evaluating a query for a given topic. In an example, learned parameters 340 include levels or concentrations of certain GIT microbiota to are indicative of a pathogen risk, animal performance (e.g., animal weight, size, nutrition level, etc.) or pathogen risk.

Profiles 345 can include learned information, such as groups of one or more biomarkers, that are useful for obtaining information from GIT biological samples for training the models 305 or for operating the trained models 330. In an example profiles include learned biomarkers that are useful for selecting primers or probes for fabricating array chips for obtaining GIT microbiota for operating a model based on a selected or associated topic. In an example, the profiles 345 are obtained or extracted from the trained models 330. In a more specific example, the profiles 345 include biomarkers selected from one or more feature vectors of the trained models 330.

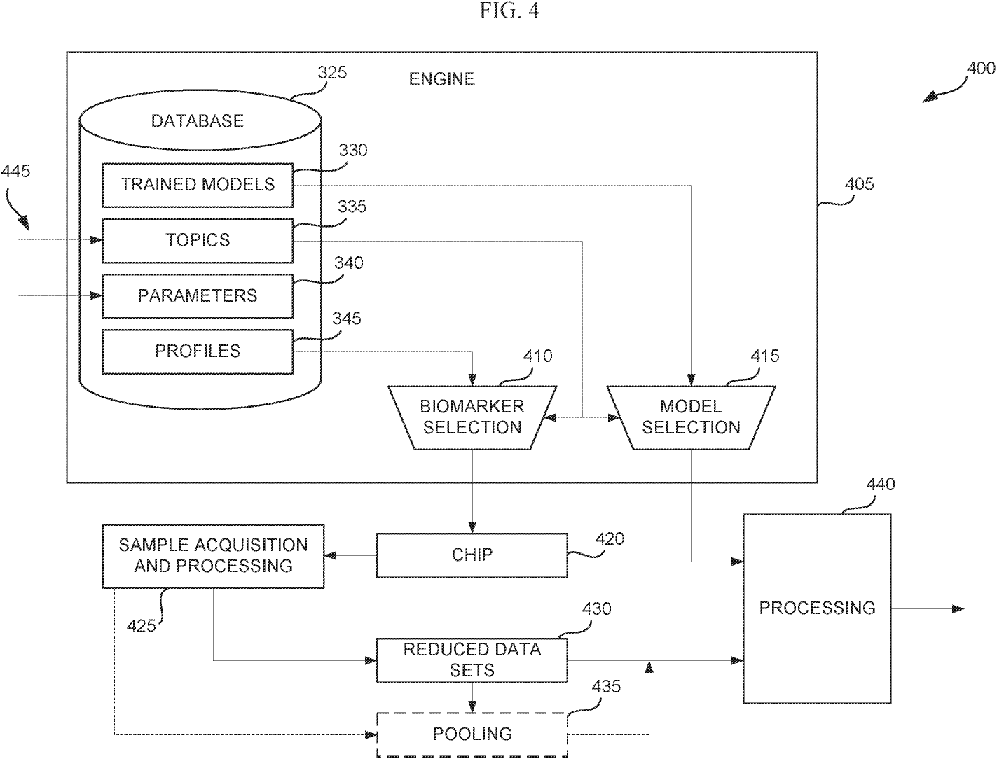

FIG. 4 is a diagram illustrating an example of a system 400 for using an engine 405 (e.g., a model engine) to process GIT microbiota data using machine learning, according to various embodiments. The system 400 can include one or more components for performing the operations of, or that correspond to, components of any other the figures described herein. The system 400 includes the engine 405, array chip 420, sample acquisition component 425, data sets 430, and processing component 440.

The engine 405 can include a database 325, a biomarker selection component 410 and a selection component 415.

The biomarker selection component 410 can be configured to select, based on a selected topic, such as determined by inputs 445, a set of biomarkers from profiles 345 for fabricating an array chip 420.

The array chip 420 can be an example of the array chips described in the discussing of FIGS. 1-3 and may be configured to obtain GIT biological data using primers or probes derived from the selected biomarkers.

Sample acquisition and processing component 425 can be an example of the sample acquisition component 110 and the sample processing component 115. Sample acquisition and processing component 425 can be configured to obtain or generate reduced data sets 420 from GIT biological samples. The data sets are reduced relative to data sets that would otherwise be obtained using micro array chips that are not fabricated using the set of biomarkers that are specifically selected to obtain information for operating the trained models 330. In some examples, sample pooling techniques can be used to reduce the number of samples obtained needed to operate a selected model.

In an example, a first number of GIT samples (e.g., a first sample size) can be used to generate the trained models 330, while a second smaller number of GIT samples (e.g., a second sample size) can be used to operate the trained models 330 for clustering, classification, or predictive operations. In such an example, the second number of samples are obtained for similar animals at different farm sites and are combined or aggregated in sample acquisition and processing component 425 or in reduced data sets 430.

The model selection component 415 can be configured to select, such as based on a selected topic 335, one or more of the trained models 330 for generating clustering, classification, or predictive outputs based on the reduced data sets 430.

The processing component 440 can include a circuit or software application that is configured to operate or execute a select trained model for generating clustering, classification, or predictive outputs based on the reduced data sets 430.

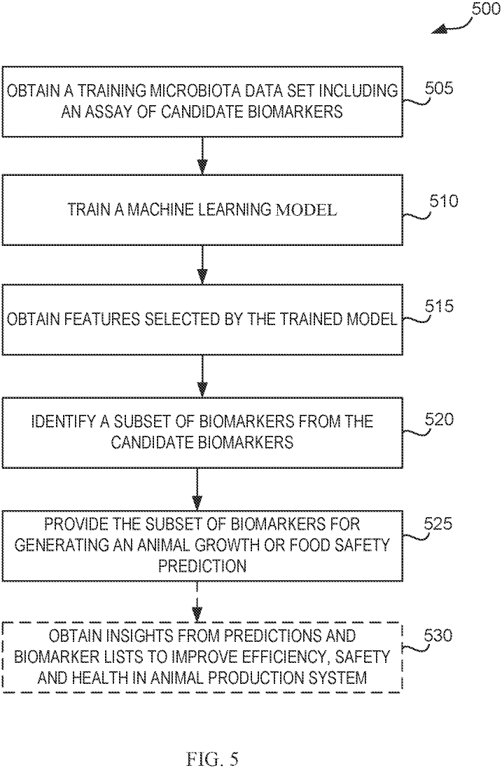

FIG. 5 depicts an example of a process 500 for obtaining an engine, such as the engine 405, to identify biomarkers for predicting food safety or animal growth, according to various embodiments. The process 500 can be an implementation of one or more of the techniques described herein. At 505, a microbiota data set is obtained. In an example, the microbiota data set is obtained from an assay of GIT biological samples having candidate biomarkers, as described herein. The candidate data set can also be obtained from supplementary data sources such as a farm site or a feed processing or production facility. At 510, one or more machine learning techniques are used to train or generate a clustering, classification, or prediction engine, such as the clustering engine 135, classification engine 140, or prediction engine 145 as shown in FIG. 1. At 515, one or more features can be obtained from the selected models. Such features can include any individual measurable property or characteristic of the microbiota data set obtained 505 that is used in the execution or evaluation of the models trained at 510. In an example the features include learned information that is indicative of a set of biomarkers that are used by the trained models to generate a classification or a prediction. At 520, the set of biomarkers can be obtained or selected from the set of candidate biomarkers based on the selected features. At 525, the selected set of biomarkers are provided for generating an animal performance or food safety classification or prediction. At 530, the machine learning techniques or the trained or generated clustering, classification, or prediction engines can be used to obtain insights from predictions and biomarker (e.g., biomarker lists) to improve efficiency, safety and health in animal production system.

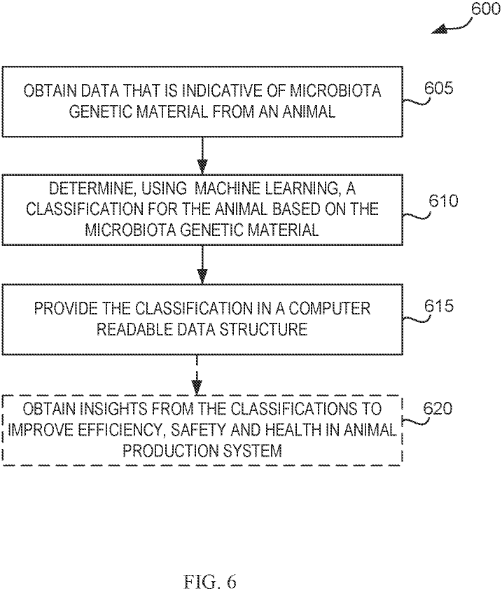

FIG. 6 depicts an example of a process 600 for using an engine to predict the performance or pathogen risk of an animal based on GIT microbiota, according to various embodiments. The process 600 can be an implementation of one or more of the techniques described herein. At 605, data that is indicative of GIT microbiota genetic material from an animal is obtained, such as by using any of the techniques described herein. In an example, the data is obtained from a number of animals (e.g., 6-24 animals) so as obtain statistically meaningful data. At 610, a classification or predication for the animal is determined using data obtained from the microbiota genetic material and one or more machine learning models. At 615, the classification is provided in a computer readable data structure, such as a digital file, or a formatted web page. At 620, insights can be obtained from the classifications or predictions to improve efficiency, safety and health in animal production system.

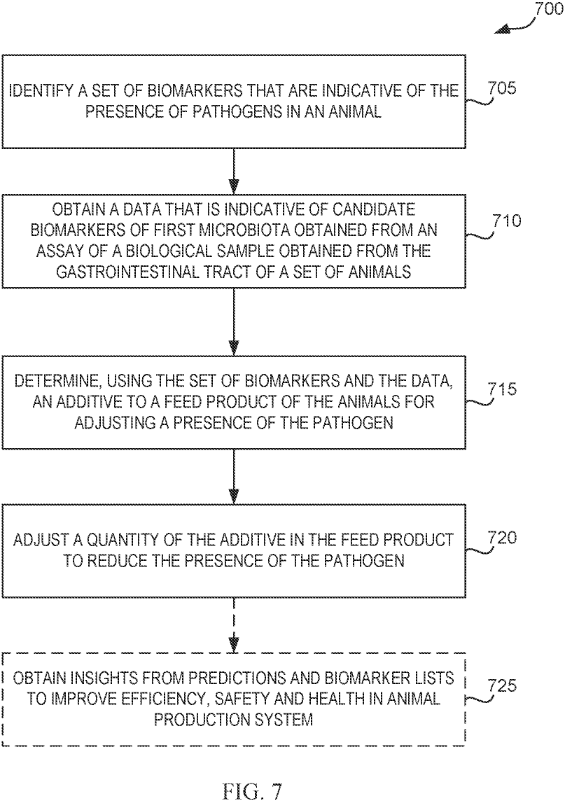

FIG. 7 depicts an example of a process 700 for using a microbiota engine to control the performance or foot safety risk of an animal based on GIT microbiota, according to various embodiments. The process 700 can be an implementation of one or more of the techniques described herein. At 705, a set of biomarkers that are indicative of the presence of pathogens in an animal can be identified. At 710, data that is indicative of a candidate biomarkers of a set of microbiota is obtained from an assay of a biological material obtained from a GIT biological sample of a set of animals.

At 715, the set of biomarkers and the data are used to determine or identify an additive to a feed product that can be provided to the animals to adjust the presence of the pathogens in the GIT of the animals. In an example, the additive includes an ingredient or a nutrient that can be added to existing feed products that are provided to the animals. In another example, the additive includes an ingredient or a nutrient that can be used to generate new feed products for provision to the animals. Identifying the additive can include providing the feed product, which includes a first quantity of the additive, to the animals. Identifying the additive can include can determining, based on the set of biomarkers, a first presence of the pathogen in GITs of the animals and providing the adjusted feed product having a second quantity of the additive to the animals. Identifying the additive can then include determining, based on the set of biomarkers, a second presence of the pathogen in gastrointestinal tracts of the animals and identifying a difference between the first presence and the second presence of pathogen.

At 720, the quantity of the additive in the feed product can be adjusted to reduce the presence of the pathogen in the GIT of the animal.

At 725, machine learning techniques or trained or generated clustering, classification, or prediction engines can be used to obtain insights from predictions and biomarker (e.g., biomarker lists) to improve efficiency, safety and health in animal production system.

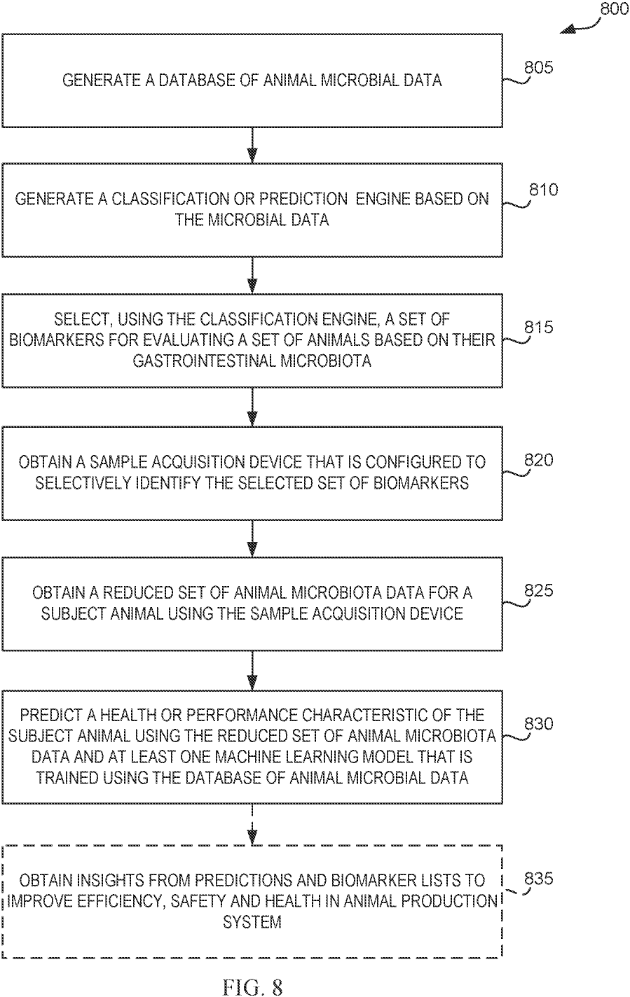

FIG. 8 depicts an example of a process 800 for operating a system to control animal performance or pathogen risk using machine learning and GIT microbiota data, according to various embodiments. The process 800 can be an implementation of one or more of the techniques described herein. At 805, a database of animal microbial data, such as GIT microbiota data and associated analysis and supplementary information, can be generated. At 810, a classification or prediction engine can be generated based on animal microbial data, such as by using any of the machine learning techniques or models described herein. At 815, the classification or prediction engine can be used to select or identify a set of biomarkers for evaluating a set of one or more animals based on their GIT microbiota. At 820, a sample acquisition device, such as the array chips described herein, can be obtained or fabricated based on the selected or identified biomarkers. At 825, a reduced set of animal microbial data for a subject set of one or more animals can be obtained using the sample acquisition device. At 830, a health or performance characteristic of the subject set of animals can be predicted using the reduced set of animal microbiota data and at least one machine learning model that is trained using the database of animal microbial data.

At 835, machine learning techniques or trained or generated clustering, classification, or prediction engines can be used to obtain insights from predictions and biomarker (e.g., biomarker lists) to improve efficiency, safety and health in animal production system.

The processes described in the discussion herein can include any other steps or operations for implementing the techniques described herein.

While the operations processes described in the discussion FIGS. 5-8 are shown as happening sequentially in a specific order, in other examples, one or more of the operations may be performed in parallel or in a different order. Additionally, one or more operations may be repeated two or more times.

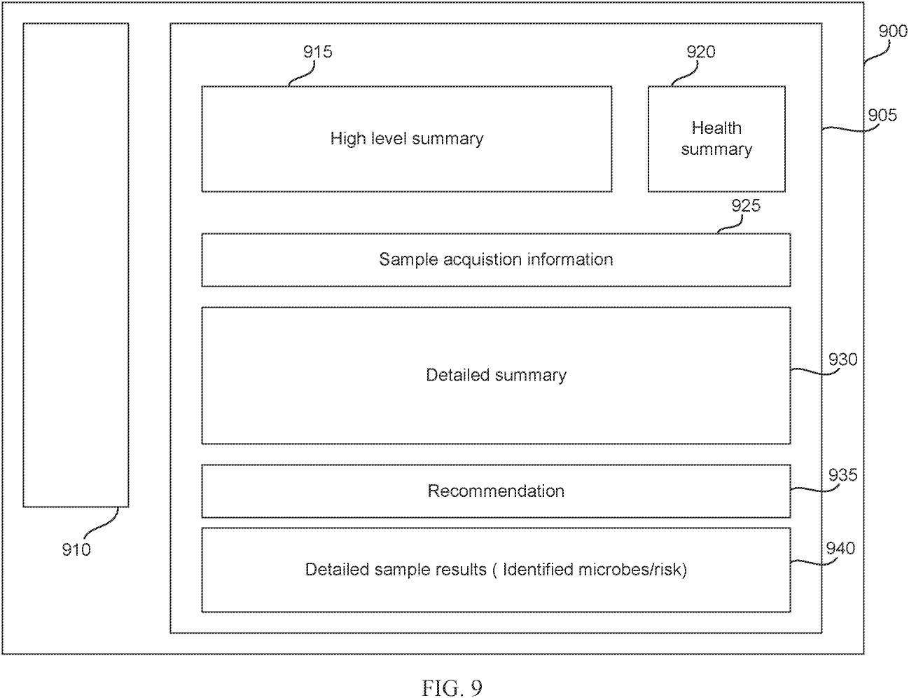

FIG. 9 is a diagram illustrating an example of a user interface 900 reporting performance or pathogen risk of animals based on GIT microbiota, according to various embodiments. In an example, the interface 900 is a component of the reporting component 150, as shown in FIG. 1. In an example, the user interface 900 is a graphical user interface to a web or internet server or a software application that is configured to provide information that is associated with performance or pathogen risk of the animals. The user interface 900 can include a reporting area 905 that is configured to provide (e.g., display or render) the performance or pathogen risk information and a navigation area 910 that is configured to enable a user to navigate between one or more report, summary, or other information page.

The reporting area 905 can include a first summary area 915 and a health summary area 920. The first summary or recommendation area 915 can provide descriptive summary of the overall health of the animals as inferred from the GIT microbiota, while the health summary area 920 can provide a graphical indicator, such as a dial, or health of the animals. The reporting area 905 can further include a sample information area 925 for reporting information associated with the sample acquisition process, such as the date, time, location of the sample acquisition. The reporting area 905 can further include detailed summary area 930, such as to provide a detailed summary of salient microbiota identified in a sample and the pathogen risk or performance impact of the presence of the identified microbiota. The reporting area 905 can further include a recommendation area 935, such as for providing recommendations based on the identified microbiota and associated pathogen risk and performance impacts. The reporting area 905 can additionally include a detailed results area 940, such as for providing information associated with of each taxa of microbiota identified in the sample.

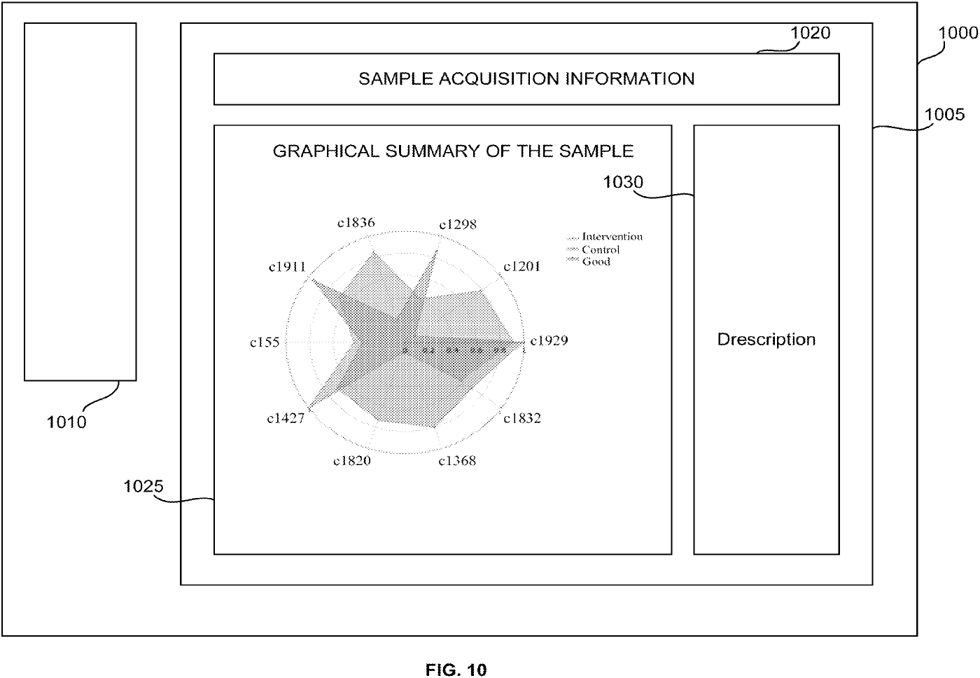

FIG. 10 is a diagram illustrating an example of a user interface 1000 reporting a graphical data that is indicative of performance or pathogen risk based on GIT microbiota obtained from GIT biological samples of a set of animals, according to various embodiments. In an example, the interface 1000 is a component of the reporting component 150, as shown in FIG. 1, or a component of the user interface 900, as shown in FIG. 9. In an example, the user interface 1000 is a graphical user interface to a web or internet server or a software application that is configured to provide information that is associated with animal performance or pathogen risk. The user interface 1000 can include a reporting area 1005 that is configured to provide (e.g., display or render) the animal performance or pathogen risk information and a navigation area 1010 that is configured to enable a user to navigate between one or more report, summary, or other information page or interface. The reporting area 1005 can further include a sample information area 1025 for reporting information associated with the sample acquisition process, such as the date, time, location of the sample acquisition. The reporting area 1005 can further include a graphical summary area 1025. In an example, the graphical summary area 1025 can provide a graphical summary of the relative presence (e.g., population) of salient microbiota in a GIT biological sample. Such summary can also include a graphical display of a good or recommended distribution of microbiota. The reporting area 905 can include a description area, such as for providing information about the microbiota displayed in the graphical summary area 1025.

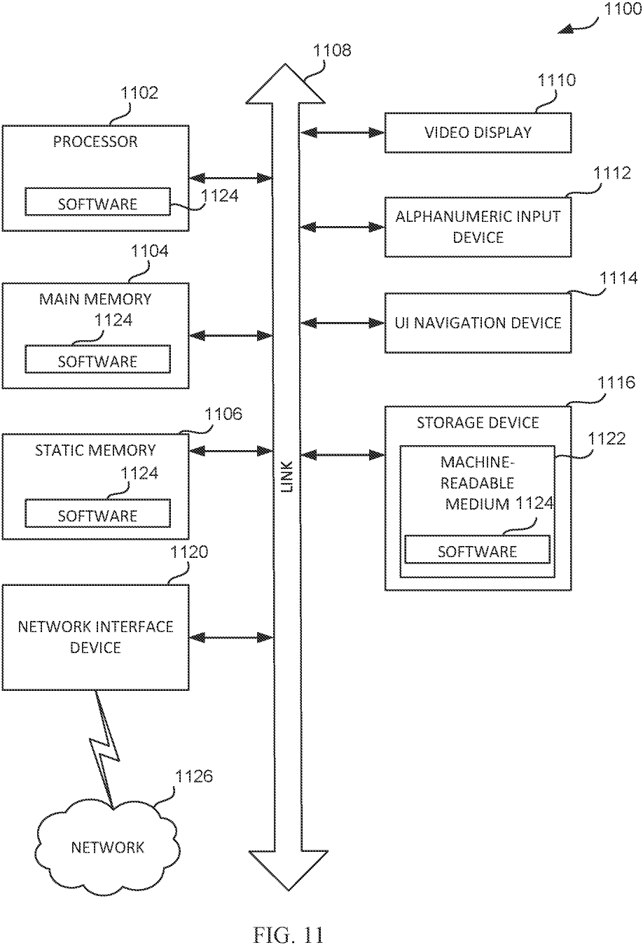

FIG. 11 illustrates a diagrammatic representation of a machine in the form of a computer system within which a set of instructions may be executed for causing the machine to perform any one or more of the methodologies discussed herein, according to an example of the present disclosure. The computer system 1100 is an example of one or more of the computing resources discussed herein.

In alternative examples, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a vehicle subsystem, a personal computer (PC), a tablet PC, a hybrid tablet, a personal digital assistant (PDA), a mobile telephone, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1100 includes at least one processor 1102 (e.g., a central processing unit (CPU), a graphics processing unit (GPU) or both, processor cores, compute nodes, etc.), a main memory 1104 and a static memory 1106, which communicate with each other via a link 1108 (e.g., bus). The computer system 1100 may further include a video display unit 1110, an alphanumeric input device 1112 (e.g., a keyboard), and a user interface (UI) navigation device 1114 (e.g., a mouse). In one example, the video display unit 1110, input device 1112 and UI navigation device 1114 are incorporated into a touch screen display. The computer system 1100 may additionally include a storage device 1116 (e.g., a drive unit), such as a global positioning system (GPS) sensor, compass, accelerometer, gyrometer, magnetometer, or other sensors.

The storage device 1116 includes a machine-readable medium 1122 on which is stored one or more sets of data structures and instructions 1124 (e.g., software) embodying or utilized by any one or more of the methodologies or functions described herein. In an example, the one or more instructions 1124 can constitute the processing component 125, clustering engine 135, classification engine 140, prediction engine 145, reporting component 150, database 130 or 325, training component 320, or applications for implementing the processes described in FIGS. 5-8, or the analysis application 460, as described herein. The instructions 1124 may also reside, completely or at least partially, within the main memory 1104, static memory 1106, and/or within the processor 1102 during execution thereof by the computer system 1100, with the main memory 1104, static memory 1106, and the processor 1102 also constituting machine-readable media.

While the machine-readable medium 1122 is illustrated in an example to be a single medium, the term “machine-readable medium” may include a single medium or multiple media (e.g., a centralized or distributed database, and/or associated caches and servers) that store the one or more instructions 1124. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1124 may further be transmitted or received over a communications network 1126 using a transmission medium via the network interface device 1120 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Bluetooth, Wi-Fi, 3G, and 4G LTE/LTE-A, 5G, DSRC, or WiMAX networks). The term “transmission medium” shall be taken to include any intangible medium that is capable of storing, encoding, or carrying instructions for execution by the machine, and includes digital or analog communications signals or other intangible medium to facilitate communication of such software.

Embodiments may be implemented in one or a combination of hardware, firmware, and software. Embodiments may also be implemented as instructions stored on a machine-readable storage device, which may be read and executed by at least one processor to perform the operations described herein. A machine-readable storage device may include any non-transitory mechanism for storing information in a form readable by a machine (e.g., a computer). For example, a machine-readable storage device may include read-only memory (ROM), random-access memory (RAM), magnetic disk storage media, optical storage media, flash-memory devices, and other storage devices and media.

A processor subsystem may be used to execute the instruction on the -readable medium. The processor subsystem may include one or more processors, each with one or more cores. Additionally, the processor subsystem may be disposed on one or more physical devices. The processor subsystem may include one or more specialized processors, such as a graphics processing unit (GPU), a digital signal processor (DSP), a field programmable gate array (FPGA), or a fixed function processor.

Examples, as described herein, may include, or may operate on, logic or a number of components, modules, or mechanisms. Modules may be hardware, software, or firmware communicatively coupled to one or more processors in order to carry out the operations described herein. Modules may be hardware modules, and as such modules may be considered tangible entities capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software; the general-purpose hardware processor may be configured as respective different modules at different times. Software may accordingly configure a hardware processor, for example, to constitute a particular module at one instance of time and to constitute a different module at a different instance of time. Modules may also be software or firmware modules, which operate to perform the methodologies described herein.

Circuitry or circuits, as used in this document, may comprise, for example, singly or in any combination, hardwired circuitry, programmable circuitry such as computer processors comprising one or more individual instruction processing cores, state machine circuitry, and/or firmware that stores instructions executed by programmable circuitry. The circuits, circuitry, or modules may, collectively or individually, be embodied as circuitry that forms part of a larger system, for example, an integrated circuit (IC), system on-chip (SoC), desktop computers, laptop computers, tablet computers, servers, smart phones, etc.

As used in any example herein, the term “logic” may refer to firmware and/or circuitry configured to perform any of the aforementioned operations. Firmware may be embodied as code, instructions or instruction sets and/or data that are hard-coded (e.g., nonvolatile) in memory devices and/or circuitry.

### Various Examples

Example 1 is a method of training a microbiota model engine to identify biomarkers for predicting food safety or animal growth, the method comprising: obtaining first data that is indicative of an assay of candidate biomarkers obtained from material from gastrointestinal tracts of a set of animals, the assay performed at specified intervals in the lifecycle of the set of animals, the set of animals manifesting specified characteristics at the specified intervals; training the microbiota model engine using the first data to generate a prediction based on at least one of a food safety or an animal growth criterion; obtaining, from the trained microbiota model engine, a set of features used by the microbiota model engine to generate the prediction; identifying a subset of biomarkers from amongst the candidate biomarkers from the set of features; and providing the subset of biomarkers for generating food safety or animal growth predictions.

In Example 2, the subject matter of Example 1 includes, wherein providing the subset of biomarkers for generating food safety or animal growth predictions comprises: storing the subset of biomarkers in a database comprising records that associate one or more sets of biomarker with a food safety or animal growth topic.

In Example 3, the subject matter of Examples 1-2 includes, wherein the specified characteristics comprise body mass and training the machine learning model using the first data to generate the prediction comprises: training the machine learning model to predict the body mass of animals.

In Example 4, the subject matter of Examples 1-3 includes, wherein the biomarkers comprise a profile of one or more bacteria or other microbiota.

In Example 5, the subject matter of Examples 1-4 includes, wherein the prediction comprises a predicted food safety risk based the probable presence of specified bacteria in the gastrointestinal tract of the animal.

In Example 6, the subject matter of Examples 1-5 includes, wherein training a machine learning model using the first data to generate a prediction comprises: obtaining second data comprising a subset of the first data that was obtained within an specified interval of time during the lifecycle of the set of animals, the interval selected to improve the likelihood or accuracy of the prediction of the trained machine learning model; and training the machine learning model using the second data.

Example 7 is a method comprising: obtaining first data that is indicative of genetic material of first microbiota obtained from a gastrointestinal tract of an animal at specified intervals in the lifecycle of the animal; determining, based on the first data and using a first microbiota model engine, a model for the animal, the first microbiota model engine trained using supervised learning and data obtained from gastrointestinal tracts of two or more animals; and providing the classification in a computer readable data structure for display on a graphical user interface.

In Example 8, the subject matter of Example 7 includes, wherein obtaining the first data comprises: processing gastrointestinal samples obtained from the animal using an intestinal flora chip, the intestinal flora chip being configured to generate genetic information that is indicative of a predetermined subset of the total microbiota obtained from the gastrointestinal tract of the animal.

In Example 9, the subject matter of Example 8 includes, wherein the predetermined subset of the total microbiota is selected using a second microbiota classification engine, the second microbiota model engine being trained using the total microbiota obtained from gastrointestinal tracts of a second set of two or more animals.

In Example 10, the subject matter of Examples 7-9 includes, wherein determining the model for the animal comprises generating a prediction of a nutritional content of the animal, the nutrient content being indicative of the presence or deficiency of one or more nutrients.

In Example 11, the subject matter of Examples 7-10 includes, wherein determining the model for the animal comprises generating a prediction of a body mass of the animal.

In Example 12, the subject matter of Examples 7-11 includes, wherein determining the model for the animal comprises: generating a prediction of a concentration of second microbiota in gastrointestinal tract of the animal; and determining, based on the prediction, an adjustment to a feed product or other nutrient to provide to the animal to improve at least one of a body mass of the animal or a food safety risk of the animal.

In Example 13, the subject matter of Examples 7-12 includes, wherein determining the model for the animal comprises: generating a prediction of a concentration of second microbiota in gastrointestinal tract of the animal; and determining, based on the prediction, an adjustment to a feed product provided to the animal to adjust the microbiota of the animal, wherein the adjustment is selected to improve the likelihood that an offspring of the animal will have an specified body mass or microbiota concentration.

In Example 14, the subject matter of Examples 7-13 includes, wherein determining the model for the animal comprises: generating a prediction of a concentration of second microbiota in gastrointestinal tract of the animal; and determine, based on the prediction, a likelihood that the animal is food safety risk.

In Example 15, the subject matter of Examples 7-14 includes, wherein determining the model for the animal comprises generating a prediction of a concentration of second microbiota in gastrointestinal tract of the animal, and the method further comprises: identifying a feed product that is associated with the second microbiota; and determining, based on the classification and the identifying, an adjustment to an additive or nutrient of the feed product to increase or decrease a concentration of the second microbiota in the animal.

Example 16 is a method of reducing antibiotic usage to control the presence of a pathogen in a population of animals, the method comprising: determining, using a microbiota model engine that is stored in the memory of a computing system, a set of biomarkers from gastrointestinal tracts of the animals that are indicative of the presence of the pathogen; obtaining first data that is indicative of assay of candidate biomarkers of first microbiota obtained from a gastrointestinal tract of an animal at specified intervals in the lifecycle of the animal; identifying, using the set of biomarkers and the first data, an additive to a feed product of the animals for adjusting a presence of the pathogen; and adjusting a quantity of the additive in the feed product to reduce the presence of the pathogen.

In Example 17, the subject matter of Example 16 includes, wherein identifying the additive comprises: providing the feed product with a first quantity of the additive to the animals; determining, based on the set of biomarkers, a first presence of the pathogen in gastrointestinal tracts of the animals; providing the feed product with a second quantity of the additive to the animals; determining, based on the set of biomarkers, a second presence of the pathogen in gastrointestinal tracts of the animals; and identifying a difference between the first presence and the second presence of pathogen.

Example 18 is a graphical user interface (GUI) to report a sample analysis, the GUI comprising: a first area to report a summary of the analysis; and a second area to report a graphical categorical metric associated with the summary of the analysis.

Example 19 is a graphical user interface (GUI) to report a sample analysis of a population of animals, the GUI comprising: a first area to report a current distribution of microbes in a population; a second to report a predicted distribution of microbes in the population; and a third to report a financial impact associated with the current or predicted microbial distribution.

In Example 20, the subject matter of Example 19 includes, a fourth area to report adjustable metrics and predictions associated with the distribution of microbes, the fourth area comprising categorical indicators associated with the adjustable metrics.

Example 21 is a system of training a microbiota model engine to identify biomarkers for predicting food safety or animal growth, the system comprising: hardware processing circuitry; a hardware memory, comprising instructions that when executed configure the hardware processing circuitry to perform operations comprising: obtaining first data that is indicative of an assay of candidate biomarkers obtained from material from gastrointestinal tracts of a set of animals, the assay performed at specified intervals in the lifecycle of the set of animals, the set of animals manifesting specified characteristics at the specified intervals; training the microbiota model engine using the first data to generate a prediction based on at least one of a food safety or an animal growth criterion; obtaining, from the trained microbiota model engine, a set of features used by the microbiota model engine to generate the prediction; identifying a subset of biomarkers from amongst the candidate biomarkers from the set of features; and providing the subset of biomarkers for generating food safety or animal growth predictions.

In Example 22, the subject matter of Example 21 includes, the operations further comprising: storing the subset of biomarkers in a database comprising records that associate one or more sets of biomarker with a food safety or animal growth topic.

In Example 23, the subject matter of Examples 21-22 includes, wherein the specified characteristics comprise body mass and the operations further comprising: training the machine learning model to predict the body mass of animals.

In Example 24, the subject matter of Examples 21-23 includes, wherein the biomarkers comprise a profile of one or more bacteria or other microbiota.

In Example 25, the subject matter of Examples 21-24 includes, wherein the prediction comprises a predicted food safety risk based the probable presence of specified bacteria in the gastrointestinal tract of the animal.

In Example 26, the subject matter of Examples 21-25 includes, the operations further comprising: obtaining second data comprising a subset of the first data that was obtained within an specified interval of time during the lifecycle of the set of animals, the interval selected to improve the likelihood or accuracy of the prediction of the trained machine learning model; and training the machine learning model using the second data.

Example 27 is a non-transitory computer readable storage medium comprising instructions that when executed configure hardware processing circuitry to perform operations for training a microbiota model engine to identify biomarkers for predicting food safety or animal growth, the operations comprising: obtaining first data that is indicative of an assay of candidate biomarkers obtained from material from gastrointestinal tracts of a set of animals, the assay performed at specified intervals in the lifecycle of the set of animals, the set of animals manifesting specified characteristics at the specified intervals; training the model classification engine using the first data to generate a prediction based on at least one of a food safety or an animal growth criterion; obtaining, from the trained microbiota model engine, a set of features used by the microbiota model engine to generate the prediction; identifying a subset of biomarkers from amongst the candidate biomarkers from the set of features; and providing the subset of biomarkers for generating food safety or animal growth predictions.

In Example 28, the subject matter of Example 27 includes, the operations further comprising: storing the subset of biomarkers in a database comprising records that associate one or more sets of biomarker with a food safety or animal growth topic.

In Example 29, the subject matter of Examples 27-28 includes, wherein the specified characteristics comprise body mass and the operations further comprising: training the machine learning model to predict the body mass of animals.

In Example 30, the subject matter of Examples 27-29 includes, wherein the biomarkers comprise a profile of one or more bacteria or other microbiota.

In Example 31, the subject matter of Examples 27-30 includes, wherein the prediction comprises a predicted food safety risk based the probable presence of specified bacteria in the gastrointestinal tract of the animal.

In Example 32, the subject matter of Examples 27-31 includes, the operations further comprising: obtaining second data comprising a subset of the first data that was obtained within an specified interval of time during the lifecycle of the set of animals, the interval selected to improve the likelihood or accuracy of the prediction of the trained machine learning model; and training the machine learning model using the second data.

Example 33 is a system comprising: hardware processing circuitry; a hardware memory, comprising instructions that when executed configure the hardware processing circuitry to perform operations comprising: obtaining first data that is indicative of genetic material of first microbiota obtained from a gastrointestinal tract of an animal at specified intervals in the lifecycle of the animal; determining, based on the first data and using a first microbiota classification engine, a classification for the animal, the first microbiota model engine trained using supervised learning and data obtained from gastrointestinal tracts of two or more animals; and providing the model in a computer readable data structure for display on a graphical user interface.

In Example 34, the subject matter of Example 33 includes, the operations further comprising: processing gastrointestinal samples obtained from the animal using an intestinal flora chip, the intestinal flora chip being configured to generate genetic information that is indicative of a predetermined subset of the total microbiota obtained from the gastrointestinal tract of the animal.

In Example 35, the subject matter of Example 34 includes, the operations further comprising: selecting the predetermined subset of the total microbiota using a second microbiota model engine, wherein the second microbiota model engine is trained using the total microbiota obtained from gastrointestinal tracts of a second set of two or more animals.

In Example 36, the subject matter of Examples 33-35 includes, the operations further comprising generating a prediction of a nutritional content of the animal, the nutrient content being indicative of the presence or deficiency of one or more nutrients.

In Example 37, the subject matter of Examples 33-36 includes, the operations further comprising generating a prediction of a body mass of the animal.

In Example 38, the subject matter of Examples 33-37 includes, the operations further comprising: generating a prediction of a concentration of second microbiota in gastrointestinal tract of the animal; and determining, based on the prediction, an adjustment to a feed product or other nutrient to provide to the animal to improve at least one of a body mass of the animal or a food safety risk of the animal.

In Example 39, the subject matter of Examples 33-38 includes, the operations further comprising: generating a prediction of a concentration of second microbiota in gastrointestinal tract of the animal; and determining, based on the prediction, an adjustment to a feed product provided to the animal to adjust the microbiota of the animal, wherein the adjustment is selected to improve the likelihood that an offspring of the animal will have an specified body mass or microbiota concentration.

In Example 40, the subject matter of Examples 33-39 includes, the operations further comprising: generating a prediction of a concentration of second microbiota in gastrointestinal tract of the animal; and determine, based on the prediction, a likelihood that the animal is food safety risk.

In Example 41, the subject matter of Examples 33-40 includes, the operations further comprising: generating a prediction of a concentration of second microbiota in gastrointestinal tract of the animal; and identifying a feed product that is associated with the second microbiota; and determining, based on the model and the identifying, an adjustment to an additive or nutrient of the feed product to increase or decrease a concentration of the second microbiota in the animal.

Example 42 is a non-transitory computer readable storage medium comprising instructions that when executed configure hardware processing circuitry to perform operations comprising: obtaining first data that is indicative of genetic material of first microbiota obtained from a gastrointestinal tract of an animal at specified intervals in the lifecycle of the animal; determining, based on the first data and using a first microbiota model engine, a classification for the animal, the first microbiota model engine trained using supervised learning and data obtained from gastrointestinal tracts of two or more animals; and providing the model in a computer readable data structure for display on a graphical user interface.

In Example 43, the subject matter of Example 42 includes, the operations further comprising: processing gastrointestinal samples obtained from the animal using an intestinal flora chip, the intestinal flora chip being configured to generate genetic information that is indicative of a predetermined subset of the total microbiota obtained from the gastrointestinal tract of the animal.

In Example 44, the subject matter of Example 43 includes, the operations further comprising: selecting the predetermined subset of the total microbiota using a second microbiota model engine, wherein the second microbiota model engine is trained using the total microbiota obtained from gastrointestinal tracts of a second set of two or more animals.

In Example 45, the subject matter of Examples 42-44 includes, the operations further comprising generating a prediction of a nutritional content of the animal, the nutrient content being indicative of the presence or deficiency of one or more nutrients.

In Example 46, the subject matter of Examples 42-45 includes, the operations further comprising generating a prediction of a body mass of the animal.

In Example 47, the subject matter of Examples 42-46 includes, the operations further comprising: generating a prediction of a concentration of second microbiota in gastrointestinal tract of the animal; and determining, based on the prediction, an adjustment to a feed product or other nutrient to provide to the animal to improve at least one of a body mass of the animal or a food safety risk of the animal.

In Example 48, the subject matter of Examples 42-47 includes, the operations further comprising: generating a prediction of a concentration of second microbiota in gastrointestinal tract of the animal; and determining, based on the prediction, an adjustment to a feed product provided to the animal to adjust the microbiota of the animal, wherein the adjustment is selected to improve the likelihood that an offspring of the animal will have a specified body mass or microbiota concentration.

In Example 49, the subject matter of Examples 42-48 includes, the operations further comprising: generating a prediction of a concentration of second microbiota in gastrointestinal tract of the animal; and determine, based on the prediction, a likelihood that the animal is food safety risk.

In Example 50, the subject matter of Examples 42-49 includes, the operations further comprising: generating a prediction of a concentration of second microbiota in gastrointestinal tract of the animal; and identifying a feed product that is associated with the second microbiota; and determining, based on the classification and the identifying, an adjustment to an additive or nutrient of the feed product to increase or decrease a concentration of the second microbiota in the animal.

Example 51 is a system of reducing antibiotic usage to control the presence of a pathogen in a population of animals, the system comprising: hardware processing circuitry; a hardware memory, comprising instructions that when executed configure the hardware processing circuitry to perform operations comprising: determining, using a microbiota model engine that is stored in the memory of a computing system, a set of biomarkers from gastrointestinal tracts of the animals that are indicative of the presence of the pathogen; obtaining first data that is indicative of assay of candidate biomarkers of first microbiota obtained from a gastrointestinal tract of an animal at specified intervals in the lifecycle of the animal; identifying, using the set of biomarkers and the first data, an additive to a feed product of the animals for adjusting a presence of the pathogen; and adjusting a quantity of the additive in the feed product to reduce the presence of the pathogen.

In Example 52, the subject matter of Example 51 includes, the operations further comprising: providing the feed product with a first quantity of the additive to the animals; determining, based on the set of biomarkers, a first presence of the pathogen in gastrointestinal tracts of the animals; providing the feed product with a second quantity of the additive to the animals; determining, based on the set of biomarkers, a second presence of the pathogen in gastrointestinal tracts of the animals; and identifying a difference between the first presence and the second presence of pathogen.

Example 53 is a non-transitory computer readable storage medium comprising instructions that when executed configure hardware processing circuitry to perform operations for reducing antibiotic usage to control the presence of a pathogen in a population of animals, the operations comprising: determining, using a microbiota model engine that is stored in the memory of a computing system, a set of biomarkers from gastrointestinal tracts of the animals that are indicative of the presence of the pathogen; obtaining first data that is indicative of assay of candidate biomarkers of first microbiota obtained from a gastrointestinal tract of an animal at specified intervals in the lifecycle of the animal; identifying, using the set of biomarkers and the first data, an additive to a feed product of the animals for adjusting a presence of the pathogen; and adjusting a quantity of the additive in the feed product to reduce the presence of the pathogen.

In Example 54, the subject matter of Example 53 includes, the operations further comprising: providing the feed product with a first quantity of the additive to the animals; determining, based on the set of biomarkers, a first presence of the pathogen in gastrointestinal tracts of the animals; providing the feed product with a second quantity of the additive to the animals; determining, based on the set of biomarkers, a second presence of the pathogen in gastrointestinal tracts of the animals; and identifying a difference between the first presence and the second presence of pathogen.

Example 55 is a method for generating a graphical user interface (GUI) to report a sample analysis, the GUI comprising: rendering a first area to report a summary of the analysis; and rendering a second area to report a graphical categorical metric associated with the summary of the analysis.

Example 56 is a non-transitory computer readable storage medium comprising instructions that when executed configure hardware processing circuitry to perform operations for generating a graphical user interface (GUI) to report a sample analysis, the operations comprising: rendering a first area to report a summary of the analysis; and rendering a second area to report a graphical categorical metric associated with the summary of the analysis.

Example 57 is a non-transitory computer readable storage medium comprising instructions that when executed configure hardware processing circuitry to perform operations for generating a graphical user interface (GUI) to report a sample analysis of a population of animals, the operations comprising: rendering a first area to report a current distribution of microbes in a population; rendering a second to report a predicted distribution of microbes in the population; and rendering a third to report a financial impact associated with the current or predicted microbial distribution.

In Example 58, the subject matter of Example 57 includes, operations further comprising: rendering a fourth area to report adjustable metrics and predictions associated with the distribution of microbes, the fourth area comprising categorical indicators associated with the adjustable metrics.

Example 59 is at least one machine-readable medium including instructions that, when executed by processing circuitry, cause the processing circuitry to perform operations to implement of any of Examples 1-58.

Example 60 is an apparatus comprising means to implement of any of Examples 1-58.

Example 61 is a system to implement of any of Examples 1-58.

Example 62 is a method to implement of any of Examples 1-58.

The above detailed description includes references to the accompanying drawings, which form a part of the detailed description. The drawings show, by way of illustration, specific examples that may be practiced. These examples are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other examples may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as examples may feature a subset of said features. Further, examples may include fewer features than those disclosed in a particular example. Thus, the following claims are hereby incorporated into the Detailed Description, with a claim standing on its own as a separate example. The scope of the examples disclosed herein is to be determined with reference to the appended claims, along with the full scope of equivalents to which such claims are entitled.

What is claimed is:

1. A method of training a microbiota model engine to identify biomarkers for predicting food safety or animal growth, the method comprising:
obtaining first data that is indicative of an assay of candidate biomarkers obtained from material from gastrointestinal tracts of a set of animals, the assay performed at specified intervals in the lifecycle of the set of animals, the set of animals manifesting specified characteristics at the specified intervals;
training the microbiota model engine using the first data to generate a prediction based on at least one of a food safety or an animal growth criterion;
obtaining, from the trained microbiota model engine, a set of features used by the microbiota model engine to generate the prediction;
identifying a subset of biomarkers from amongst the candidate biomarkers from the set of features; and
providing the subset of biomarkers for generating food safety or animal growth predictions.

2. The method of claim 1, wherein providing the subset of biomarkers for generating food safety or animal growth predictions comprises:
storing the subset of biomarkers in a database comprising records that associate one or more sets of biomarker with a food safety or animal growth topic.

3. The method of claim 1, wherein the specified characteristics comprise body mass and training the machine learning model using the first data to generate the prediction comprises:
training the machine learning model to predict the body mass of animals.

4. The method of claim 1, wherein the biomarkers comprise a profile of one or more bacteria or other microbiota.

5. The method of claim 1, wherein the prediction comprises a predicted food safety risk based the probable presence of specified bacteria in the gastrointestinal tract of the animal.

6. The method of claim 1, wherein training a machine learning model using the first data to generate a prediction comprises:
obtaining second data comprising a subset of the first data that was obtained within an specified interval of time during the lifecycle of the set of animals, the interval selected to improve the likelihood or accuracy of the prediction of the trained machine learning model; and
training the machine learning model using the second data.

7. A method comprising:
obtaining first data that is indicative of genetic material of first microbiota obtained from a gastrointestinal tract of an animal at specified intervals in the lifecycle of the animal;
determining, based on the first data and using a first microbiota model engine, a model for the animal, the first microbiota model engine trained using supervised learning and data obtained from gastrointestinal tracts of two or more animals; and
providing the classification in a computer readable data structure for display on a graphical user interface.

8. The method of claim 7, wherein obtaining the first data comprises:
processing gastrointestinal samples obtained from the animal using an intestinal flora chip, the intestinal flora chip being configured to generate genetic information that is indicative of a predetermined subset of the total microbiota obtained from the gastrointestinal tract of the animal.

9. The method of claim 8, wherein the predetermined subset of the total microbiota is selected using a second microbiota model engine, the second microbiota model engine being trained using the total microbiota obtained from gastrointestinal tracts of a second set of two or more animals.

10. The method of claim 7, wherein determining the model for the animal comprises generating a prediction of a nutritional content of the animal, the nutrient content being indicative of the presence or deficiency of one or more nutrients.

11. The method of claim 7, wherein determining the model for the animal comprises generating a prediction of a body mass of the animal.

12. The method of claim 7, wherein determining the model for the animal comprises:
generating a prediction of a concentration of second microbiota in gastrointestinal tract of the animal; and
determining, based on the prediction, an adjustment to a feed product or other nutrient to provide to the animal to improve at least one of a body mass of the animal or a food safety risk of the animal.

13. The method of claim 7, wherein determining the model for the animal comprises:
generating a prediction of a concentration of second microbiota in gastrointestinal tract of the animal; and
determining, based on the prediction, an adjustment to a feed product provided to the animal to adjust the microbiota of the animal, wherein the adjustment is selected to improve the likelihood that an offspring of the animal will have an specified body mass or microbiota concentration.

14. The method of claim 7, wherein determining the model for the animal comprises:
generating a prediction of a concentration of second microbiota in gastrointestinal tract of the animal; and
determine, based on the prediction, a likelihood that the animal is food safety risk.

15. The method of claim 7, wherein determining the model for the animal comprises generating a prediction of a concentration of second microbiota in gastrointestinal tract of the animal, and the method further comprises:
identifying a feed product that is associated with the second microbiota; and
determining, based on the model and the identifying, an adjustment to an additive or nutrient of the feed product to increase or decrease a concentration of the second microbiota in the animal.

16. A method of reducing antibiotic usage to control the presence of a pathogen in a population of animals, the method comprising:
determining, using a microbiota model engine that is stored in the memory of a computing system, a set of biomarkers from gastrointestinal tracts of the animals that are indicative of the presence of the pathogen;
obtaining first data that is indicative of assay of candidate biomarkers of first microbiota obtained from a gastrointestinal tract of an animal at specified intervals in the lifecycle of the animal;
identifying, using the set of biomarkers and the first data, an additive to a feed product of the animals for adjusting a presence of the pathogen; and
adjusting a quantity of the additive in the feed product to reduce the presence of the pathogen.

17. The method of claim 16, wherein identifying the additive comprises:
providing the feed product with a first quantity of the additive to the animals;
determining, based on the set of biomarkers, a first presence of the pathogen in gastrointestinal tracts of the animals;
providing the feed product with a second quantity of the additive to the animals;
determining, based on the set of biomarkers, a second presence of the pathogen in gastrointestinal tracts of the animals; and
identifying a difference between the first presence and the second presence of pathogen.

18. A graphical user interface (GUI) to report a sample analysis, the GUI comprising:
a first area to report a summary of the analysis; and
a second area to report a graphical categorical metric associated with the summary of the analysis.

19. A graphical user interface (GUI) to report a sample analysis of a population of animals, the GUI comprising:
a first area to report a current distribution of microbes in a population;
a second to report a predicted distribution of microbes in the population; and
a third to report a financial impact associated with the current or predicted microbial distribution.

20. The GUI of claim 19, further comprising:
a fourth area to report adjustable metrics and predictions associated with the distribution of microbes, the fourth area comprising categorical indicators associated with the adjustable metrics.

21. A system of training a microbiota model engine to identify biomarkers for predicting food safety or animal growth, the system comprising:
hardware processing circuitry;
a hardware memory, comprising instructions that when executed configure the hardware processing circuitry to perform operations comprising:
obtaining first data that is indicative of an assay of candidate biomarkers obtained from material from gastrointestinal tracts of a set of animals, the assay performed at specified intervals in the lifecycle of the set of animals, the set of animals manifesting specified characteristics at the specified intervals;
training the microbiota model engine using the first data to generate a prediction based on at least one of a food safety or an animal growth criterion;
obtaining, from the trained microbiota model engine, a set of features used by the microbiota model engine to generate the prediction;
identifying a subset of biomarkers from amongst the candidate biomarkers from the set of features; and
providing the subset of biomarkers for generating food safety or animal growth predictions.

22. The system of claim 21, the operations further comprising:
storing the subset of biomarkers in a database comprising records that associate one or more sets of biomarker with a food safety or animal growth topic.

23. The method of claim 21, wherein the specified characteristics comprise body mass and the operations further comprising:
training the machine learning model to predict the body mass of animals.

24. The system of claim 21, wherein the biomarkers comprise a profile of one or more bacteria or other microbiota.

25. The system of claim 21, wherein the prediction comprises a predicted food safety risk based the probable presence of specified bacteria in the gastrointestinal tract of the animal.

26. The system of claim 21, the operations further comprising:
obtaining second data comprising a subset of the first data that was obtained within an specified interval of time during the lifecycle of the set of animals, the interval selected to improve the likelihood or accuracy of the prediction of the trained machine learning model; and
training the machine learning model using the second data.

27. A non-transitory computer readable storage medium comprising instructions that when executed configure hardware processing circuitry to perform operations for training a microbiota model engine to identify biomarkers for predicting food safety or animal growth, the operations comprising:
obtaining first data that is indicative of an assay of candidate biomarkers obtained from material from gastrointestinal tracts of a set of animals, the assay performed at specified intervals in the lifecycle of the set of animals, the set of animals manifesting specified characteristics at the specified intervals;
training the microbiota model engine using the first data to generate a prediction based on at least one of a food safety or an animal growth criterion;
obtaining, from the trained microbiota model engine, a set of features used by the microbiota model engine to generate the prediction;
identifying a subset of biomarkers from amongst the candidate biomarkers from the set of features; and
providing the subset of biomarkers for generating food safety or animal growth predictions.

28. The non-transitory computer readable storage medium of claim 27, the operations further comprising:
storing the subset of biomarkers in a database comprising records that associate one or more sets of biomarker with a food safety or animal growth topic.

29. The non-transitory computer readable storage medium of claim 27, wherein the specified characteristics comprise body mass and the operations further comprising:
training the machine learning model to predict the body mass of animals.

30. The non-transitory computer readable storage medium of claim 27, wherein the biomarkers comprise a profile of one or more bacteria or other microbiota.

31. The non-transitory computer readable storage medium of claim 27, wherein the prediction comprises a predicted food safety risk based the probable presence of specified bacteria in the gastrointestinal tract of the animal.

32. The non-transitory computer readable storage medium of claim 27, the operations further comprising:
obtaining second data comprising a subset of the first data that was obtained within an specified interval of time during the lifecycle of the set of animals, the interval selected to improve the likelihood or accuracy of the prediction of the trained machine learning model; and
training the machine learning model using the second data.

33. A system comprising:
hardware processing circuitry;
a hardware memory, comprising instructions that when executed configure the hardware processing circuitry to perform operations comprising:
obtaining first data that is indicative of genetic material of first microbiota obtained from a gastrointestinal tract of an animal at specified intervals in the lifecycle of the animal;
determining, based on the first data and using a first microbiota model engine, a model for the animal, the first microbiota model engine trained using supervised learning and data obtained from gastrointestinal tracts of two or more animals; and
providing the model in a computer readable data structure for display on a graphical user interface.

34. The system of claim 33, the operations further comprising:
processing gastrointestinal samples obtained from the animal using an intestinal flora chip, the intestinal flora chip being configured to generate genetic information that is indicative of a predetermined subset of the total microbiota obtained from the gastrointestinal tract of the animal.

35. The system of claim 34, the operations further comprising:
selecting the predetermined subset of the total microbiota using a second microbiota model engine, wherein the second microbiota model engine is trained using the total microbiota obtained from gastrointestinal tracts of a second set of two or more animals.

36. The system of claim 33, the operations further comprising generating a prediction of a nutritional content of the animal, the nutrient content being indicative of the presence or deficiency of one or more nutrients.

37. The system of claim 33, the operations further comprising generating a prediction of a body mass of the animal.

38. The system of claim 33, the operations further comprising:
generating a prediction of a concentration of second microbiota in gastrointestinal tract of the animal; and
determining, based on the prediction, an adjustment to a feed product or other nutrient to provide to the animal to improve at least one of a body mass of the animal or a food safety risk of the animal.

39. The system of claim 33, the operations further comprising:
generating a prediction of a concentration of second microbiota in gastrointestinal tract of the animal; and
determining, based on the prediction, an adjustment to a feed product provided to the animal to adjust the microbiota of the animal, wherein the adjustment is selected to improve the likelihood that an offspring of the animal will have an specified body mass or microbiota concentration.

40. The system of claim 33, the operations further comprising:
generating a prediction of a concentration of second microbiota in gastrointestinal tract of the animal; and
determine, based on the prediction, a likelihood that the animal is food safety risk.

41. The system of claim 33, the operations further comprising:
generating a prediction of a concentration of second microbiota in gastrointestinal tract of the animal; and
identifying a feed product that is associated with the second microbiota; and
determining, based on the model and the identifying, an adjustment to an additive or nutrient of the feed product to increase or decrease a concentration of the second microbiota in the animal.

42. A non-transitory computer readable storage medium comprising instructions that when executed configure hardware processing circuitry to perform operations comprising:
obtaining first data that is indicative of genetic material of first microbiota obtained from a gastrointestinal tract of an animal at specified intervals in the lifecycle of the animal;
determining, based on the first data and using a first microbiota model engine, a model for the animal, the first microbiota model engine trained using supervised learning and data obtained from gastrointestinal tracts of two or more animals; and
providing the model in a computer readable data structure for display on a graphical user interface.

43. The non-transitory computer readable storage medium of claim 42, the operations further comprising:
processing gastrointestinal samples obtained from the animal using an intestinal flora chip, the intestinal flora chip being configured to generate genetic information that is indicative of a predetermined subset of the total microbiota obtained from the gastrointestinal tract of the animal.

44. The non-transitory computer readable storage medium of claim 43, the operations further comprising:
selecting the predetermined subset of the total microbiota using a second microbiota model engine, wherein the second microbiota model engine is trained using the total microbiota obtained from gastrointestinal tracts of a second set of two or more animals.

45. The non-transitory computer readable storage medium of claim 42, the operations further comprising generating a prediction of a nutritional content of the animal, the nutrient content being indicative of the presence or deficiency of one or more nutrients.

46. The non-transitory computer readable storage medium of claim 42, the operations further comprising generating a prediction of a body mass of the animal.

47. The non-transitory computer readable storage medium of claim 42, the operations further comprising:
generating a prediction of a concentration of second microbiota in gastrointestinal tract of the animal; and
determining, based on the prediction, an adjustment to a feed product or other nutrient to provide to the animal to improve at least one of a body mass of the animal or a food safety risk of the animal.

48. The non-transitory computer readable storage medium 42, the operations further comprising:
generating a prediction of a concentration of second microbiota in gastrointestinal tract of the animal; and
determining, based on the prediction, an adjustment to a feed product provided to the animal to adjust the microbiota of the animal, wherein the adjustment is selected to improve the likelihood that an offspring of the animal will have a specified body mass or microbiota concentration.

49. The non-transitory computer readable storage medium 42, the operations further comprising:
generating a prediction of a concentration of second microbiota in gastrointestinal tract of the animal; and
determine, based on the prediction, a likelihood that the animal is food safety risk.

50. The non-transitory computer readable storage medium of claim 42, the operations further comprising:
generating a prediction of a concentration of second microbiota in gastrointestinal tract of the animal; and
identifying a feed product that is associated with the second microbiota; and
determining, based on the model and the identifying, an adjustment to an additive or nutrient of the feed product to increase or decrease a concentration of the second microbiota in the animal.

51. A system of reducing antibiotic usage to control the presence of a pathogen in a population of animals, the system comprising:
hardware processing circuitry;
a hardware memory, comprising instructions that when executed configure the hardware processing circuitry to perform operations comprising:
determining, using a microbiota model engine that is stored in the memory of a computing system, a set of biomarkers from gastrointestinal tracts of the animals that are indicative of the presence of the pathogen;
obtaining first data that is indicative of assay of candidate biomarkers of first microbiota obtained from a gastrointestinal tract of an animal at specified intervals in the lifecycle of the animal;
identifying, using the set of biomarkers and the first data, an additive to a feed product of the animals for adjusting a presence of the pathogen; and
adjusting a quantity of the additive in the feed product to reduce the presence of the pathogen.

52. The system of claim 51, the operations further comprising:
providing the feed product with a first quantity of the additive to the animals;
determining, based on the set of biomarkers, a first presence of the pathogen in gastrointestinal tracts of the animals;
providing the feed product with a second quantity of the additive to the animals;
determining, based on the set of biomarkers, a second presence of the pathogen in gastrointestinal tracts of the animals; and
identifying a difference between the first presence and the second presence of pathogen.

53. A non-transitory computer readable storage medium comprising instructions that when executed configure hardware processing circuitry to perform operations for reducing antibiotic usage to control the presence of a pathogen in a population of animals, the operations comprising:
determining, using a microbiota model engine that is stored in the memory of a computing system, a set of biomarkers from gastrointestinal tracts of the animals that are indicative of the presence of the pathogen;
obtaining first data that is indicative of assay of candidate biomarkers of first microbiota obtained from a gastrointestinal tract of an animal at specified intervals in the lifecycle of the animal;
identifying, using the set of biomarkers and the first data, an additive to a feed product of the animals for adjusting a presence of the pathogen; and
adjusting a quantity of the additive in the feed product to reduce the presence of the pathogen.

54. The non-transitory computer readable storage medium of claim 53, the operations further comprising:
providing the feed product with a first quantity of the additive to the animals;
determining, based on the set of biomarkers, a first presence of the pathogen in gastrointestinal tracts of the animals;
providing the feed product with a second quantity of the additive to the animals;
determining, based on the set of biomarkers, a second presence of the pathogen in gastrointestinal tracts of the animals; and
identifying a difference between the first presence and the second presence of pathogen.

55. A method for generating a graphical user interface (GUI) to report a sample analysis, the GUI comprising:
rendering a first area to report a summary of the analysis; and
rendering a second area to report a graphical categorical metric associated with the summary of the analysis.

56. A non-transitory computer readable storage medium comprising instructions that when executed configure hardware processing circuitry to perform operations for generating a graphical user interface (GUI) to report a sample analysis, the operations comprising:
rendering a first area to report a summary of the analysis; and
rendering a second area to report a graphical categorical metric associated with the summary of the analysis.

57. A non-transitory computer readable storage medium comprising instructions that when executed configure hardware processing circuitry to perform operations for generating a graphical user interface (GUI) to report a sample analysis of a population of animals, the operations comprising:
rendering a first area to report a current distribution of microbes in a population;
rendering a second to report a predicted distribution of microbes in the population; and
rendering a third to report a financial impact associated with the current or predicted microbial distribution.

58. The method of claim 57, operations further comprising:
rendering a fourth area to report adjustable metrics and predictions associated with the distribution of microbes, the fourth area comprising categorical indicators associated with the adjustable metrics.
